(19) United States
(12) Patent Application Publication
(10) Pub. No.: US 2006/0217400 A1
(43) Pub. Date: Sep. 28, 2006

(54) Triazolopyrimidines, methods for the production thereof, use thereof for controlling harmful fungi, and substances containing said triazolopyrimidines

(76) Inventors: Bernd Müller, (US); Jordi Tormo i Blasco, Laudenbach (DE); Thomas Grote, Wachenheim (DE); Carsten Blettner, Mannheim (DE); Markus Gewehr, Kastellaun (DE); Wassilios Grammenos, Ludwigshafen (DE); Andreas Gypser, Mannheim (DE); Joachim Rheinheimer, Ludwigshafen (DE); Peter Schäfer, Ottersheim (DE); Frank Schieweck, Hessheim (DE); Anja Schwögler, Mannheim (DE); Eberhard Ammermann, Heppenheim (DE); Siegfried Strathmann, Limburgerhof (DE); Ulrich Schöfl, Brühl (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 10/539,559

(22) PCT Filed: Dec. 17, 2003
(86) PCT No.: PCT/EP03/14374

§ 371 Date: Mar. 21, 2006

(30) Foreign Application Priority Data
Dec. 20, 2002 (DE) 10261189.0

(51) Int. Cl. A01N 43/90; C07D 487/04

(52) U.S. Cl. 514/259.31; 544/262

(57) ABSTRACT

Triazolopyrimidines of the formula I 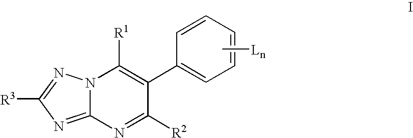
in which the index n and the substituents R¹, R² and R³ are as defined in the description, and processes for preparing these compounds, compositions comprising them and their use for controlling harmful fungi are described.

The present invention relates to triazolopyrimidines of the formula I 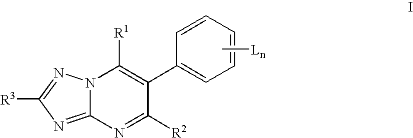
in which the index and the substituents are as defined below:
n is 0 or an integer from 1 5 5;
L is halogen, cyano, hydroxy, cyanato (OCN), C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, a five- to ten-membered saturated, partially unsaturated or aromatic heterocycle which contains 1 to 4 heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
   m is 0, 1 or 2;
   A,A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl, phenyl, naphthyl, or a five- to ten-membered saturated, partially unsaturated or aromatic heterocycle which is attached via carbon and which contains one to four heteroatoms from the group consisting of O, N and S,
where L and/or R¹ may be partially or fully halogenated or may be substituted by one to four identical or different groups R^{a}:
   R^{a} is halogen, cyano, C₁-C₆-alkyl, C₃-C₆-cycloalkyl, C₅-C₆-cycloalkenyl, C₁-C₆-alkoxy, C₁-C₆-alkylthio, C₂-C₆-alkenyl, C₂-C₆-alkenyloxy, C₃-C₆-alkynyloxy, C₂-C₁₀-alkynyl, phenyl, naphthyl, a five- to ten-membered saturated, partially unsaturated or aromatic heterocycle which contains one to four heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
where these aliphatic, alicyclic or aromatic groups for their part may be partially or fully halogenated or may carry one to three groups R^{b}:
   R^{b} is halogen, cyano, nitro, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups mentioned contain 2 to 8 carbon atoms in these radicals;
   and/or one to three of the following radicals:
   cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups; and
R² is C₁-C₄-alkyl, C₂-C₄-alkenyl or C₂-C₄-alkynyl, which may be substituted by halogen, cyano, nitro, C₁-C₂-alkoxy or C₁-C₄-alkoxycarbonyl; and also cyano, chlorine, methoxy;
R³ is halogen, cyano, C₁-C₈-alkyl, C₁-C₄-haloalkyl, hydroxy, C₁-C₈-alkoxy, C₃-C₈-alkenyl, C₃-C₈-alkenyloxy, C₁-C₈-haloalkoxy, C₃-C₈-haloalkenyloxy, C₃-C₈-cycloalkyl, N(A′)A, N(A′)-C(═O)-A or S(═O)ₘ-A.

Moreover, the invention relates to processes and intermediates for preparing these compounds, to compositions comprising them and to their use for controlling phytopathogenic harmful fungi.

Triazolopyrimidines substituted in the 2-position are disclosed in EP-A 71 792, EP-A 141 317, WO 02/88126 and WO 02/88127. The compounds described in the publications mentioned are suitable for controlling harmful fungi.

However, in many cases their activity is unsatisfactory.

It is an object of the present invention to provide compounds having improve activity and/or a broader activity spectrum.

We have found that this object is achieved by the compounds defined at the outset. Furthermore, we have found processes and intermediates for their preparation, compositions comprising them and methods for controlling phytopathogenic harmful fungi using the compounds I.

The compounds of the formula I differ from those in the abovementioned publications by the substituents in the 5- and 7-positions on the triazolopyrimidine ring.

Compared to the known compounds, the compounds of the formula I have increased activity against phytopathogenic harmful fungi.

The compounds according to the invention can be obtained by different routes. Advantageously, they are obtained by reacting sulfones of the formula I′ under basic conditions with compounds of the formula II. Depending on the nature of the group R³, compounds II are either a) cyanides, hydroxides, alkoxides or amines. In these cases, the cation M in formula II is usually an ammonium, tetraalkylammonium, alkali metal or alkaline earth metal ion. In case b), where R³ is alkyl or, in particular, fluoroalkyl, M is a metal ion of valency Y, such as, for example, B, Si, Zn, Mg or Sn.

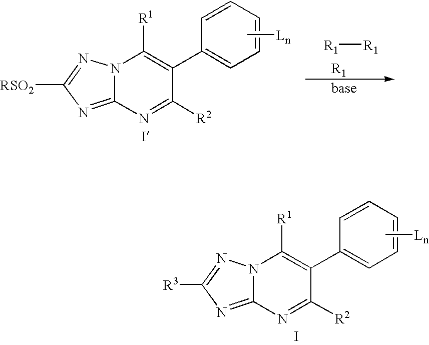

In case a), the reaction is usually carried out at temperatures from −20° C. to 120° C., preferably of from 0° C. to 25° C., in an inert organic solvent in the presence of a base. [cf. Heteroat. Chem. p. 313 (2000)].

Suitable solvents are aliphatic or aromatic hydrocarbons, such as benzene, toluene, o-, m- and p-xylene, halogenated hydrocarbons, ethers, such as diethyl ether, diisopropyl ether, tert-butyl methyl ether, dioxane, anisole and tetrahydrofuran, nitriles, ketones, alcohols and also dimethyl sulfoxide, dimethylformamide and dimethylacetamide, particularly preferably dimethyl sulfoxide, dioxane and benzene. It is also possible to use mixtures of the solvents mentioned.

Suitable bases are, in general, inorganic compounds, such as alkali metal and alkaline earth metal hydrides, such as lithium hydride, sodium hydride, potassium hydride and calcium hydride, moreover organic bases, for example tertiary amines, and also bicyclic amines. Particular preference is given to sodium hydride. The bases are generally employed in catalytic amounts; however, they can also be employed in equimolar amounts, in excess or, if appropriate, as solvents.

The starting materials are generally reacted with one another in equimolar amounts. In terms of yield, it may be advantageous to employ an excess of II, based on I′.

The preferred thiols of the formula I″ a can also be obtained from the sulfones I′. The reaction is carried out similarly as described above using an alkali metal sulfide or alkaline earth metal sulfide or alkali metal hydrogen sulfide or alkaline earth metal hydrogen sulfide or using hydrogen sulfide in the presence of bases. Sodium sulfide and sodium hydrogen sulfide have been found to be particularly suitable for the reaction. The free SH compounds can be obtained from the thiolate salts using acid.

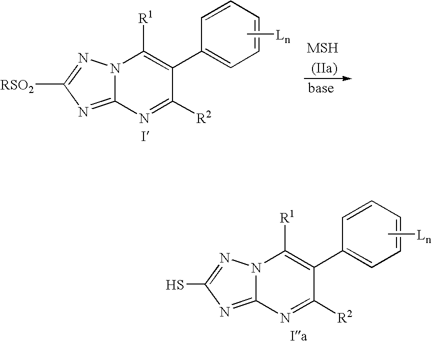

In case b), the reaction can be carried out, for example, analogously to the following methods: J. Chem. Soc., Perkin Trans. 1, 1187 (1994), ibid. 2345 (1996); WO-A 99/41255; Aust. J. Chem., 43 (1990), 733; J. Org. Chem., 43 (1978), 358; J. Chem. Soc., Chem. Commun. 866 (1979); Tetrahedron Lett., 34 (1993), 8267; ibid. 33 (1992), 413. In certain cases, it may be advantageous to carry out the reaction with Pd or Ni catalysis.

Sulfones of the formula I′ are preferably obtained by oxidizing the corresponding thio compounds I″. In formula I″, the variables are as defined under formula I. These compounds are prepared under conditions disclosed in WO 02/88127. In particular hydrogen peroxide and peracids of organic carboxylic acids have been found to be suitable oxidizing agents. However, the oxidation can also be carried out using, for example, selenium dioxide.

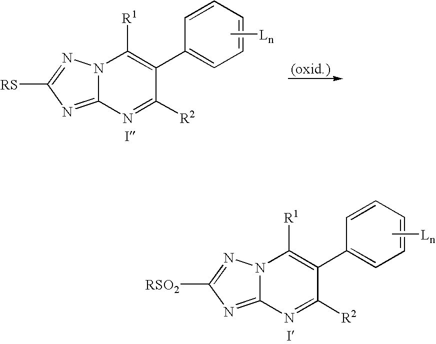

Compounds of the formula I in which R³ is C₁-C₈-alkyl, C₁-C₄-haloalkyl, SH or C₁-C₆-alkylthio can advantageously be obtained by the following synthesis route:

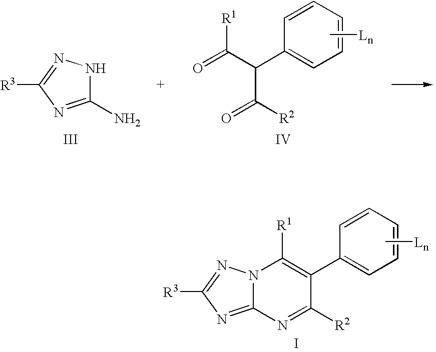

This reaction is usually carried out at 80° C.-250° C., preferably 120° C.-180° C., without solvent or in an inert organic solvent in the presence of a base [cf. EP-A 770 615] or in the presence of acetic acid under the conditions known from Adv. Het. Chem. 57 (1993), 81ff.

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide. The reaction is particularly preferably carried out without solvent or in chlorobenzene, xylene, dimethyl sulfoxide, N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, diisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines, such as diisopropylethylamine, tributylamine, N-methylmorpholine or N-methylpiperidine.

The bases are generally employed in catalytic amounts; however, they can also be used in equimolar amounts, in excess or, if appropriate, as solvent.

The starting materials are generally reaction with one another in equimolar amounts. In terms of yield, it may be advantageous to employ an excess of the base and the diketone IV, based on III.

The aminotriazoles of the formula III are disclosed, for example, in EP-A 71 792, EP-A 141 317, WO 02/088126 and WO 02/088127. 3-Methylthio-5-aminotriazole is also commercially available. The dicarbonyl compounds of the formula IV are disclosed in the literature or can be prepared by analogous processes (J. Am. Chem. Soc. 122 (2000), 1360; Org. Lett. 2 (2000), 1045; Synthetic Commun. 17 (1987), 393; Bull. Soc. Chim. Fr. 3 (1987), 438; Tetrahedron Lett. 30 (1989), 1373; J. Med. Chem. 35 (1992), 931; Tetrahedron 48 (1992), 6909; J. Org. Chem. 44 (1979), 4021.

A particularly interesting embodiment of the abovementioned process starts from triazoles of the formula IIIa 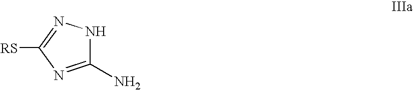
in which R is hydrogen, C₁-C₆-alkyl or unsubstituted or substituted phenyl, which are reacted with dicarbonyl compounds of the formula IV 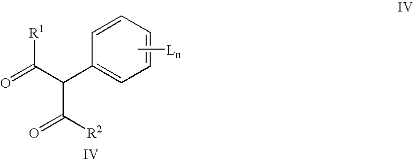
in which n, L, R¹ and R² are as defined in claim 1, to give triazolopyrimidine sulfides of the formula I″ 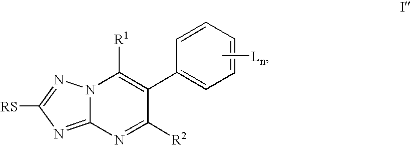
followed, if appropriate, by oxidation of I″ to sulfones of the formula I′. If appropriate, as discussed above, the sulfonyl radical can also be exchanged for other radicals R³.

The compounds of the formula I according to the invention in which R³ is SH can be prepared by reacting a 3-thio-5-aminotriazole of the formula III′ in which Π is hydrogen or a protective group which can be removed under acidic or, preferably, under basic conditions, such as methyl, unsubstituted or substituted phenyl, benzyl, in particular p-acetoxybenzyl [cf. Greene, Protective Groups in Organic Chemistry, J. Wiley & Sons, pp. 195-217 (1981); J. Org. Chem. 43 (1978), 1197] using appropriately substituted phenylmalonates of the formula IV

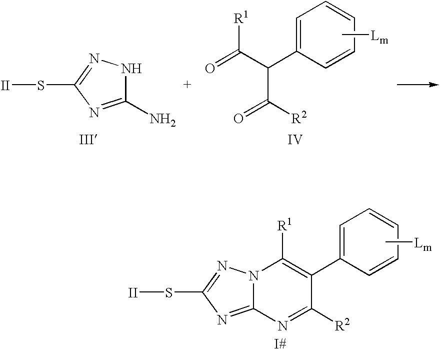

The condensation can be carried out analogously to the process described above.

3-Thio-5-aminotriazoles of the formula II are known, and some of them are commercially available. The introduction of the protective group Π into compound III′ and the removal of the protective group Π from the intermediate I# can be carried out analogously to the processes described in Greene, Protective Groups in Organic Chemistry, J. Wiley & Sons, 195-217 (1981).

A further possibility of preparing the compounds I is shown in the scheme below: 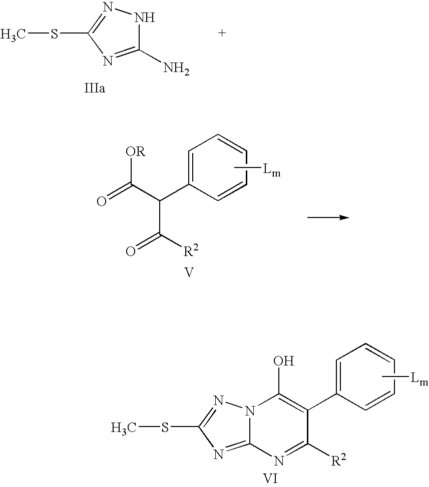
Starting with the keto esters V, the 5-alkyl-7-hydroxy-6-phenyltriazolopyrimidines VI are obtained. In formula V, R² is C₁-C₄-alkyl or C₁-C₄-haloalkyl. Using the easily obtainable 2-phenylacetoacetates (V where R²═CH₃), the 5-methyl-7-hydroxy-6-phenyltriazolopyrimidines are obtained [cf. Chem. Pharm. Bull. 9 (1961), 801]. The preparation of the starting materials V is advantageously carried out under the conditions described in EP-A 10 02 788].

The 5-alkyl-7-hydroxy-6-phenyltriazolopyrimidines obtained in this way are reacted with halogenating agents to give 7-halotriazolopyrimidines. Preference is given to using chlorinating or brominating agents such as phosphorus oxybromide, phosphorus oxychloride, thionyl chloride, thionyl bromide or sulfuryl chloride. The reaction can be carried out in the absence or in the presence of a solvent. Customary reaction temperatures are 0-150° C. or, preferably, 80-125° C. [cf. EP-A 770 615].

Using organometallic B, Zn, Mg or Sn compounds, the appropriate radical R² can be introduced into the 7-position of the 5-alkyl-7-halo-6-phenyltriazolopyrimidines obtained in this way. In some cases, it may be advantageous to carry out the reaction under Pd or Ni catalysis. The reaction can be carried out, for example, analogously to the following methods: J. Chem. Soc., Perkin Trans. 1, 1187 (1994), ibid. 2345 (1996); WO-A 99/41255; Aust. J. Chem. 43 (1990), 733; J. Org. Chem. 43 (1978), 358; J. Chem. Soc., Chem. Commun. 866 (1979); Tetrahedron Lett. 34 (1993), 8267; ibid. 33 (1992), 413.

The compounds of the formula I according to the invention can also be obtained by reacting 5-halotriazolopyrimidines of the formula VII with substituted malonic esters of the formula VIII in which R^{x} is C₁-C₄-alkyl, allyl, phenyl or benzyl, followed by hydrolysis of the resulting ester IX and decarboxylation of the carboxylic acid IXa.

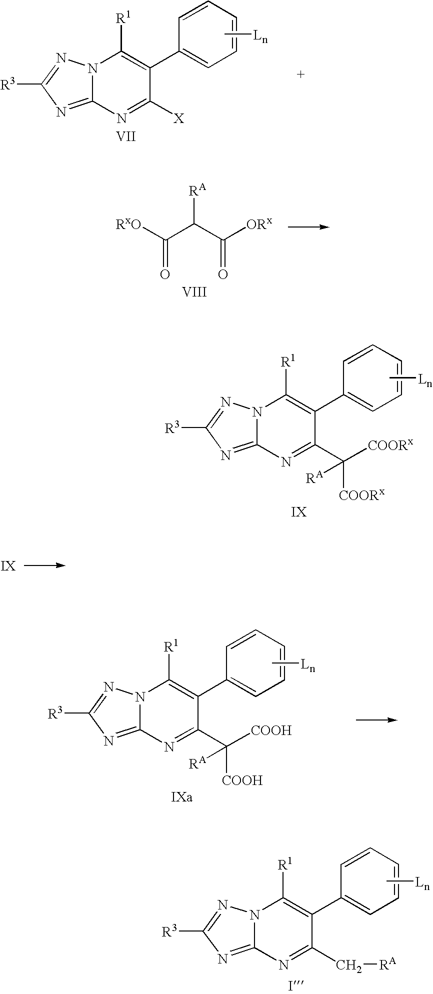

In formula VII, X is halogen, in particular chlorine or bromine. The compounds VII are known from the publications cited at the outset. In formula I′″, n, R and R¹ are as defined under formula I and and R^{A} is hydrogen or C₁-C₃-alkyl which may be substituted by halogen, cyano, nitro or C₁-C₂-alkoxy.

In a preferred embodiment of the process according to the invention, R^{A} is hydrogen or methyl, in particular hydrogen.

The starting materials VIII are known from the literature [J. Am. Chem. Soc. 64 (1942), 2714; J. Org. Chem. 39 (1974), 2172; Helv. Chim. Acta 61 (1978), 1565], or they can be prepared according to the literature cited.

The subsequent cleavage of the ester is carried out under the generally customary conditions [cf.: Greene & Wuts, Protective Groups in Organic Synthesis, Wiley (1991), p. 224 ff: Cleavage of alkyl esters under Pd catalysis (p. 248); hydrogenolysis of benzyl esters (p. 251); cleavage of methyl or ethyl esters in the presence of lithium salts, such as LiI (p. 232), LiBr or LiCl; or under acidic or alkaline conditions]. Depending on the structural elements R^{A}, Rₙ and R¹, alkaline or acidic hydrolysis of the compounds IX may be advantageous. Complete or partial decarboxylation to I′″ may already occur under the conditions of the ester hydrolysis.

The decarboxylation is usually carried out at 20° C.-180° C., preferably 50° C.-120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

A further route to the compounds of the formula I is described in the sections below:

Starting with 3-thiomethyl-5-aminotriazole of the formula IIIa, the dihydroxytriazolopyrimidines XI are prepared using appropriately substituted phenylmalonates of the formula X in which R is alkyl, preferably C₁-C₆-alkyl, in particular methyl or ethyl. Analogously, starting with 3-alkyl-5-amino-substituted triazoles, it is possible to prepare the corresponding 2-alkyl-substituted dihydroxytriazolopyrimidines.

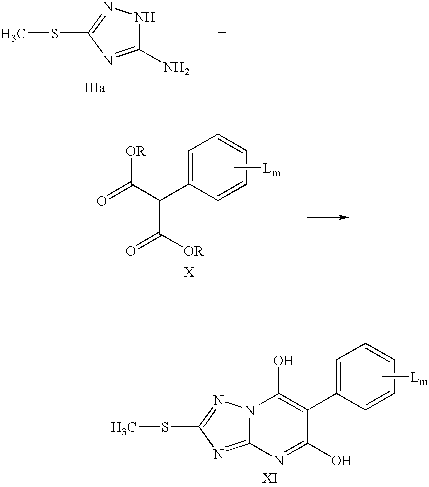

This reaction is usually carried out at 80° C.-250° C., preferably 120° C.-180° C., in the absence of a solvent or in an inert organic solvent in the presence of a base [cf. EP-A 770 615] or in the presence of acetic acid under the conditions known from Adv. Het. Chem. 57 (1993), 81ff.

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide. With particular preference, the reaction is carried out in the absence of a solvent or in chlorobenzene, xylene, dimethyl sulfoxide, N-methylpyrrolidone. It is also possible to use mixtures of the solvents mentioned.

Suitable bases are, in particular, organic bases, for example tertiary amines, such as trimethylamine, triethylamine, diisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines such as diisopropylethylamine, tributylamine, N-methylmorpholine or N-methylpiperidine. The bases are generally employed in catalytic amounts; however, they can also be used in equimolar amounts, in excess or, if appropriate, as solvent.

The starting materials are generally reacted with one another in equimolar amounts. In terms of yield, it may be advantageous to employ an excess of the base and the malonate XV, based on the thiazole XIV.

3-Thiomethyl-5-aminotriazole of the formula IIIa is commercially available. Phenylmalonates of the formula X are advantageously obtained by reacting appropriately substituted bromobenzenes with dialkyl malonates under Cu(I) catalysis [cf. Chemistry Letters (1981), 367-370; EP-A 10 02 788].

The dihydroxytriazolopyrimidines of the formula XI are converted into the dihalopyrimidines of the formula XII using the conditions disclosed in WO 94/20502. The halogenating agent used is advantageously a chlorinating agent or a brominating agent, such as phosphorus oxybromide or phosphorus oxychloride, if appropriate in the presence of a solvent.

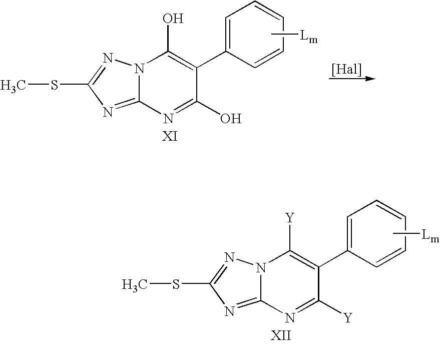

This reaction is usually carried out at 0° C.-150° C., preferably at 80° C.-120° C. [cf. EP-A 770 615].

Starting with the dihalopyrimidines of the formula XII, the substituent R¹ can be introduced into the 7-position by coupling with organometallic reagents of the formula II, giving the compounds of the formula VII. In one embodiment of this process, the reaction is carried out under transition metal catalysis, such as Ni or Pd catalysis.

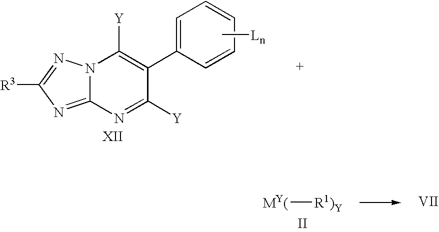

In formula II, M is a metal ion of valency Y, such as, for example, B, Zn, Mg or Sn. This reaction can be carried out, for example, analogously to the following methods: J. Chem. Soc., Perkin Trans. 1, 1187 (1994), ibid. 2345 (1996); WO-A 99/41255; Aust. J. Chem. 43 (1990), 733; J. Org. Chem. 43 (1978), 358; J. Chem. Soc., Chem. Commun. 866 (1979); Tetrahedron Lett. 34 (1993), 8267; ibid. 33 (1992), 413.

The reaction mixtures are worked up in a customary manner, for example by mixing with water, phase separation and, if required, chromatographic purification of the crude products. Some of the intermediates and end products are obtained in the form of colorless or slightly brownish, viscous oils, which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields isomer mixtures, a separation is generally not necessarily required since in some cases the individual isomers can be converted into one another during the preparation for use or upon use (for example under the action of light, acids or bases). Similar conversions may also occur after use, for example in the case of the treatment of plants in the treated plant or in the harmful fungus to be controlled.

In the definitions of the symbols given in the above formulae, collective terms were used which generally represent the following substituents:

Halogen: fluorine, chlorine, bromine and iodine;

Alkyl: saturated, straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

Haloalkyl: straight-chain or branched alkyl groups having 1 to 10 carbon atoms (as mentioned above), where all or some of the hydrogen atoms in these groups may be replaced by halogen atoms as mentioned above, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoro-2-propyl;

Alkenyl: unsaturated, straight-chain or branched hydrocarbon radicals having 2 to 4, 6, 8 or 10 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

Alkadienyl: unsaturated, straight-chain or branched hydrocarbon radicals having 4, 6, 8 or 10 carbon atoms and two double bonds in any position;

Haloalkenyl: unsaturated, straight-chain or branched hydrocarbon radicals having 2 to 10 carbon atoms and a double bond in any position (as mentioned above), where all or some of the hydrogen atoms in these groups may be replaced by halogen atoms as mentioned above, in particular by fluorine, chlorine and bromine;

Alkynyl: straight-chain or branched hydrocarbon groups having 2 to 4, 6, 8 or 10 carbon atoms and a triple bond in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

Cycloalkyl: mono- or bicyclic, saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- to ten-membered saturated, partially unsaturated or aromatic heterocycle which contains one to four heteroatoms from the group consisting of O, N and S:

5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

The scope of the present invention includes the (R)- and (S)-isomers and the racemates of compounds of the formula I having chiral centers.

Preferred embodiments of the invention are described below.

Preference is given to triazolopyrimidines of the formula I 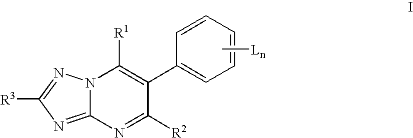
in which the index and the substituents are as defined below:
n is 0 or an integer from 1 to 5;
L is halogen, cyano, hydroxy, cyanato (OCN), C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, a five- to ten-membered saturated, partially unsaturated or aromatic heterocycle which contains 1 to 4 heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
   m is 0, 1 or 2;
   A,A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl, phenyl, naphthyl, or a five- to ten-membered saturated, partially unsaturated or aromatic heterocycle which is attached via carbon and which contains one to four heteroatoms from the group consisting of O, N and S,
where L and/or R¹ may be partially or fully halogenated or may be substituted by one to four identical or different groups R^{a}:
   R^{a} is halogen, cyano, C₁-C₆-alkyl, C₃-C₆-cycloalkyl, C₅-C₆-cycloalkenyl, C₁-C₆-alkoxy, C₁-C₆-alkylthio, C₂-C₆-alkenyl, C₂-C₆-alkenyloxy, C₃-C₆-alkynyloxy, C₂-C₁₀-alkynyl, phenyl, naphthyl, a five- to ten-membered saturated, partially unsaturated or aromatic heterocycle which contains one to four heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
   where these aliphatic, alicyclic or aromatic groups for their part may be partially or fully halogenated or may carry one to three groups R^{b}:
      R^{b} is halogen, cyano, nitro, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups mentioned contain 2 to 8 carbon atoms in these radicals;
      and/or one to three of the following radicals:
      cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups; and
R² is C₁-C₄-alkyl, C₂-C₄-alkenyl or C₂-C₄-alkynyl, which may be substituted by halogen, cyano, nitro, C₁-C₂-alkoxy or C₁-C₄-alkoxycarbonyl; and also cyano, chlorine, methoxy;
R³ is cyano, C₁-C₈-alkyl, C₁-C₄-haloalkyl, hydroxy, C₁-C₈-alkoxy, C₃-C₈-alkenyl, C₃-C₈-alkenyloxy, C₁-C₈-haloalkoxy, C₃-C₈-haloalkenyloxy, C₃-C₈-cycloalkyl, N(A′)A, N(A′)-C(═O)-A or S(═O)ₘ-A.

Triazolopyrimidines as claimed in claim 1 in which the index and the substituents are as defined below:
L is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
   m is 0, 1 or 2;
   A,A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl or a five- to ten-membered saturated, partially unsaturated or aromatic heterocycle which is attached via carbon and which contains one to four heteroatoms from the group consisting of O, N and S,
where L and/or R¹ may be partially or fully halogenated or may be substituted by one to four identical or different groups R^{a}:
   R^{a} is halogen, cyano, C₁-C₆-alkyl, C₃-C₆-cycloalkyl, C₅-C₆-cycloalkenyl, C₁-C₆-alkoxy, C₁-C₆-alkylthio, C₂-C₆-alkenyl, C₂-C₆-alkenyloxy, C₃-C₆-alkynyloxy, C₂-C₁₀-alkynyl, phenyl, naphthyl, a five- to ten-membered saturated, partially unsaturated or aromatic heterocycle which contains one to four heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
   where these aliphatic, alicyclic or aromatic groups for their part may be partially or fully halogenated or may carry one to three groups R^{b}:
      R^{b} is halogen, cyano, nitro, aminocarbonyl, aminothiocarbonyl, alkyl, haloalkyl, alkenyl, alkenyloxy, alkynyloxy, alkoxy, haloalkoxy, alkylamino, dialkylamino, alkylcarbonyl, alkylsulfonyl, alkylsulfoxyl, alkoxycarbonyl, alkylcarbonyloxy, alkylaminocarbonyl, dialkylaminocarbonyl, where the alkyl groups in these radicals contain 1 to 6 carbon atoms and the alkenyl or alkynyl groups mentioned contain 2 to 8 carbon atoms in these radicals;
      and/or one to three of the following radicals:
      cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups; and
R² is C₁-C₄-alkyl which may be substituted by halogen, cyano, nitro, C₁-C₂-alkoxy or C₁-C₄-alkoxycarbonyl.

Triazolopyrimidines in which the index and the substituents are as defined below:
R¹ is C₁-C₁₀-alkyl, C₂-C₁₀-alkenyl, C₂-C₁₀-alkynyl, C₃-C₁₂-cycloalkyl or C₃-C₁₀-cycloalkenyl,
where L and/or R¹ may be partially or fully halogenated or may be substituted by one to four identical or different groups R^{a}:
   R^{a} is halogen, cyano, C₁-C₆-alkyl, C₃-C₆-cycloalkyl, C₅-C₆-cycloalkenyl, C₁-C₆-alkoxy, C₁-C₆-alkylthio, C₂-C₆-alkenyl, C₂-C₆-alkenyloxy, C₃-C₆-alkynyloxy, C₂-C₁₀-alkynyl, —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
   where these aliphatic, alicyclic or aromatic groups for their part may be partially or fully halogenated or may carry one to three groups R^{b}:
      R^{b} is halogen, cyano, aminocarbonyl, alkyl, haloalkyl, alkenyl, alkenyloxy, alkynyloxy, alkoxy, haloalkoxy, alkylcarbonyl, alkylsulfonyl, alkylsulfoxyl, alkoxycarbonyl, alkylcarbonyloxy, alkylaminocarbonyl, dialkylaminocarbonyl, where the alkyl groups in these radicals contain 1 to 6 carbon atoms and the alkenyl or alkynyl groups mentioned in these radicals contain 2 to 8 carbon atoms;
R² is C₁-C₄-alkyl which may be substituted by halogen, cyano, nitro, C₁-C₂-alkoxy or C₁-C₄-alkoxycarbonyl.

The particularly preferred embodiments of the intermediates with respect to the variables correspond to those of the radicals Lₙ, R¹, R² and R³ of the formula I.

With a view to the intended use of the triazolopyrimidines of the formula I, the following meanings of the substituents are particularly preferred, in each case on their own or in combination:

Preference is given to compounds I in which R¹ is C₃-C₈-alkyl, C₃-C₈-alkenyl, C₃-C₈-alkynyl, C₃-C₆-cycloalkyl or C₅-C₆-cycloalkenyl.

Especially preferred are compounds I in which R¹ is C₁-C₆-alkyl or C₁-C₆-haloalkyl.

In addition, preference is given to compounds I in which R¹ is C₂-C₁₀-alkenyl or C₂-C₁₀-alkynyl.

Likewise, preference is given to compounds I in which R¹ is a 5- or 6-membered saturated or aromatic heterocycle.

Moreover, particular preference is given to compounds I in which R¹ is C₃-C₆-cycloalkyl or C₅-C₆-cycloalkenyl, which radicals may be substituted by C₁-C₄-alkyl.

Particular preference is given to compounds I in which R^{a} is halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl or C₅-C₆-cycloalkenyl, where the aliphatic or alicyclic groups for their part may be partially or fully halogenated or may carry one to three groups R^{b}.

Especially preferred are compounds I in which R^{b} is halogen, cyano, C₁-C₆-alkyl, C₂-C₆-alkenyl, C₂-C₆-alkynyl, C₁-C₆-alkylcarbonyl, C₁-C₆-haloalkylcarbonyl or C₁-C₆-alkoxy.

Particular preference is also given to compounds I in which R² is C₁-C₄-alkyl which may be substituted by halogen.

Likewise, particular preference is given to compounds I in which R² is methyl.

In addition, particular preference is given to compounds I in which R² is halomethyl.

If R¹ and/or R² contain haloalkyl or haloalkenyl groups having a center of chirality, preference is given to the (S)-isomers.

If R¹ and/or R² contain alkyl, alkenyl or alkynyl groups having a center of chirality, preference is given to the (R)-isomer.

Preference is given to compounds I in which R³ is cyano, C₅-C₈-alkyl, C₁-C₄-haloalkyl, hydroxy, C₁-C₈-alkoxy, C₃-C₈-alkenyl, C₃-C₈-alkenyloxy, C₁-C₈-haloalkoxy, C₃-C₈-haloalkenyloxy, C₃-C₈-cycloalkyl, N(A′)A, N(A′)-C(═O)-A or S(═O)ₘ-A.

Preference is also given to compounds I in which R³ is halogen and in particular fluorine or chlorine.

Furthermore, preference is given to compound I in which R³ C₁-C₄-alkyl.

Moreover, preference is given to compounds I in which R³ is N(A′)A, N(A′)-C(═O)-A or S(═O)ₘ-A.

Especially preferred are compounds I in which R³ is S(═O)ₘ-A.

Especially preferred are compounds I in which R³ is SH.

Preference is given to compounds I in which at least one group L is positioned ortho to the point of attachment with the triazolopyrimidine skeleton; in particular to those compounds in which n has the value 1, 2 or 3.

Preference is given to compounds I in which Lₙ is halogen, methyl, cyano, ethyl, C₁-haloalkyl, methoxy, —C(═O)-A, —C(═O)—O-A, —C(═O)—N(A′)A, C(A′)(═N-OA), N(A′)-C(═O)-A or S(═O)ₘ-A, where m is 0, 1 or 2 and A, A′ independently of one another are hydrogen or C₁-C₄-alkyl.

Moreover, particular preference is given to compounds I in which the phenyl group substituted by Lₙ is the group B 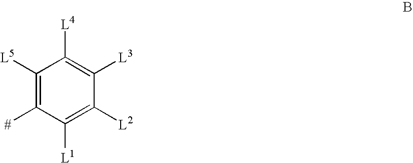
in which # denotes the point of attachment with the triazolopyrimidine skeleton and
L¹ is fluorine, chlorine, CH₃ or CF₃;
L²,L⁴ independently of one another are hydrogen, methyl or fluorine;
L³ is hydrogen, fluorine, chlorine, cyano, CH₃, SCH₃, SOCH₃, SO₂CH₃, OCH₃, NH—C(═O)CH₃, N(CH₃)—C(═O)CH₃ or COOCH₃ and
L⁵ is hydrogen, fluorine, chlorine or CH₃.

With a view to their use, special preference is given to the compounds I compiled in the tables below. Moreover, the groups mentioned in the tables for a substituent are themselves, independently of the combination in which they are mentioned, a particularly preferred embodiment of the substituents in question. 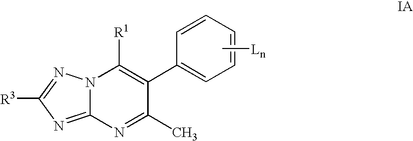
Table 1

Compounds of the formula IA in which Lₙ is 2-fluoro-6-chloro, R³ is SH and R¹ for each compound corresponds to one row of table A

Table 2

Compounds of the formula IA in which Lₙ is 2,6-difluoro, R³ is SH and R¹ for each compound corresponds to one row of table A

Table 3

Compounds of the formula IA in which Lₙ is 2,6-dichloro, R³ is SH and R¹ for each compound corresponds to one row of table A

Table 4

Compounds of the formula IA in which Lₙ is 2-fluoro-6-methyl, R³ is SH and R¹ for each compound corresponds to one row of table A

Table 5

Compounds of the formula IA in which Lₙ is 2,4,6-trifluoro, R³ is SH and R¹ for each compound corresponds to one row of table A

Table 6

Compounds of the formula IA in which Lₙ is 2,6-difluoro-4-SH, R³ is SH and R¹ for each compound corresponds to one row of table A

Table 7

Compounds of the formula IA in which Lₙ is pentafluoro, R³ is SH and R¹ for each compound corresponds to one row of table A

Table 8

Compounds of the formula IA in which Lₙ is 2-methyl-4-fluoro, R³ is SH and R¹ for each compound corresponds to one row of table A

Table 9

Compounds of the formula IA in which Lₙ is 2-trifluoromethyl, R³ is SH and R¹ for each compound corresponds to one row of table A

Table 10

Compounds of the formula IA in which Lₙ is 2-SH-6-fluoro, R³ is SH and R¹ for each compound corresponds to one row of table A

Table 11

Compounds of the formula IA in which Lₙ is 2-chloro, R³ is SH and R¹ for each compound corresponds to one row of table A

Table 12

Compounds of the formula IA in which Lₙ is 2-fluoro, R³ is SH and R¹ for each compound corresponds to one row of table A

Table 13

Compounds of the formula IA in which Lₙ is 2,4-difluoro, R³ is SH and R¹ for each compound corresponds to one row of table A

Table 14

Compounds of the formula IA in which Lₙ is 2-fluoro-4-chloro, R³ is SH and R¹ for each compound corresponds to one row of table A

Table 15

Compounds of the formula IA in which Lₙ is 2-chloro-4-fluoro, R³ is SH and R¹ for each compound corresponds to one row of table A

Table 16

Compounds of the formula IA in which Lₙ is 2,3-difluoro, R³ is SH and R¹ for each compound corresponds to one row of table A

Table 17

Compounds of the formula IA in which Lₙ is 2,5-difluoro, R³ is SH and R¹ for each compound corresponds to one row of table A

Table 18

Compounds of the formula IA in which Lₙ is 2,3,4-trifluoro, R³ is SH and R¹ for each compound corresponds to one row of table A

Table 19

Compounds of the formula IA in which Lₙ is 2-methyl, R³ is SH and R¹ for each compound corresponds to one row of table A

Table 20

Compounds of the formula IA in which Lₙ is 2,4-dimethyl, R³ is SH and R¹ for each compound corresponds to one row of table A

Table 21

Compounds of the formula IA in which Lₙ is 2-methyl-4-chloro, R³ is SH and R¹ for each compound corresponds to one row of table A

Table 22

Compounds of the formula IA in which Lₙ is 2-fluoro-4-methyl, R³ is SH and R¹ for each compound corresponds to one row of table A

Table 23

Compounds of the formula IA in which Lₙ is 2,6-dimethyl, R³ is SH and R¹ for each compound corresponds to one row of table A

Table 24

Compounds of the formula IA in which Lₙ is 2,4,6-trimethyl, R³ is SH and R¹ for each compound corresponds to one row of table A

Table 25

Compounds of the formula IA in which Lₙ is 2,6-difluoro-4-cyano, R³ is SH and R¹ for each compound corresponds to one row of table A

Table 26

Compounds of the formula IA in which Lₙ is 2,6-difluoro-4-methyl, R³ is SH and R¹ for each compound corresponds to one row of table A

Table 27

Compounds of the formula IA in which Lₙ is 2,6-difluoro-4-methoxycarbonyl, R³ is SH and R¹ for each compound corresponds to one row of table A

Table 28

Compounds of the formula IA in which Lₙ is 2-trifluoromethyl-4-fluoro, R³ is SH and R¹ for each compound corresponds to one row of table A

Table 29

Compounds of the formula IA in which Lₙ is 2-trifluoromethyl-5-fluoro, R³ is SH and R¹ for each compound corresponds to one row of table A

Table 30

Compounds of the formula IA in which Lₙ is 2-trifluoromethyl-5-chloro, R³ is SH and R¹ for each compound corresponds to one row of table A

Table 31

Compounds of the formula IA in which Lₙ is 2-fluoro-6-chloro, R³ is methylthio and R¹ for each compound corresponds to one row of table A

Table 32

Compounds of the formula IA in which Lₙ is 2,6-difluoro, R³ is methylthio and R¹ for each compound corresponds to one row of table A

Table 33

Compounds of the formula IA in which Lₙ is 2,6-dichloro, R³ is methylthio and R¹ for each compound corresponds to one row of table A

Table 34

Compounds of the formula IA in which Lₙ is 2-fluoro-6-methyl, R³ is methylthio and R¹ for each compound corresponds to one row of table A

Table 35

Compounds of the formula IA in which Lₙ is 2,4,6-trifluoro, R³ is methylthio and R¹ for each compound corresponds to one row of table A

Table 36

Compounds of the formula IA in which Lₙ is 2,6-difluoro-4-SH, R³ is methylthio and R¹ for each compound corresponds to one row of table A

Table 37

Compounds of the formula IA in which Lₙ is pentafluoro, R³ is methylthio and R¹ for each compound corresponds to one row of table A

Table 38

Compounds of the formula IA in which Lₙ is 2-methyl-4-fluoro, R³ is methylthio and R¹ for each compound corresponds to one row of table A

Table 39

Compounds of the formula IA in which Lₙ is 2-trifluoromethyl, R³ is methylthio and R¹ for each compound corresponds to one row of table A

Table 40

Compounds of the formula IA in which Lₙ is 2-methylthio-6-fluoro, R³ is methylthio and R¹ for each compound corresponds to one row of table A

Table 41

Compounds of the formula IA in which Lₙ is 2-chloro, R³ is methylthio and R¹ for each compound corresponds to one row of table A

Table 42

Compounds of the formula IA in which Lₙ is 2-fluoro, R³ is methylthio and R¹ for each compound corresponds to one row of table A

Table 43

Compounds of the formula IA in which Lₙ is 2,4-difluoro, R³ is methylthio and R¹ for each compound corresponds to one row of table A

Table 44

Compounds of the formula IA in which Lₙ is 2-fluoro-4-chloro, R³ is methylthio and R¹ for each compound corresponds to one row of table A

Table 45

Compounds of the formula IA in which Lₙ is 2-chloro-4-fluoro, R³ is methylthio and R¹ for each compound corresponds to one row of table A

Table 46

Compounds of the formula IA in which Lₙ is 2,3-difluoro, R³ is methylthio and R¹ for each compound corresponds to one row of table A

Table 47

Compounds of the formula IA in which Lₙ is 2,5-difluoro, R³ is methylthio and R¹ for each compound corresponds to one row of table A

Table 48

Compounds of the formula IA in which Lₙ is 2,3,4-trifluoro, R³ is methylthio and R¹ for each compound corresponds to one row of table A

Table 49

Compounds of the formula IA in which Lₙ is 2-methyl, R³ is methylthio and R¹ for each compound corresponds to one row of table A

Table 50

Compounds of the formula IA in which Lₙ is 2,4-dimethyl, R³ is methylthio and R¹ for each compound corresponds to one row of table A

Table 51

Compounds of the formula IA in which Lₙ is 2-methyl-4-chloro, R³ is methylthio and R¹ for each compound corresponds to one row of table A

Table 52

Compounds of the formula IA in which Lₙ is 2-fluoro-4-methyl, R³ is methylthio and R¹ for each compound corresponds to one row of table A

Table 53

Compounds of the formula IA in which Lₙ is 2,6-dimethyl, R³ is methylthio and R¹ for each compound corresponds to one row of table A

Table 54

Compounds of the formula IA in which Lₙ is 2,4,6-trimethyl, R³ is methylthio and R¹ for each compound corresponds to one row of table A

Table 55

Compounds of the formula IA in which Lₙ is 2,6-difluoro-4-cyano, R³ is methylthio and R¹ for each compound corresponds to one row of table A

Table 56

Compounds of the formula IA in which Lₙ is 2,6-difluoro-4-methyl, R³ is methylthio and R¹ for each compound corresponds to one row of table A

Table 57

Compounds of the formula IA in which Lₙ is 2,6-difluoro-4-methoxycarbonyl, R³ is methylthio and R¹ for each compound corresponds to one row of table A

Table 58

Compounds of the formula IA in which Lₙ is 2-trifluoromethyl-4-fluoro, R³ is methylthio and R¹ for each compound corresponds to one row of table A

Table 59

Compounds of the formula IA in which Lₙ is 2-trifluoromethyl-5-fluoro, R³ is methylthio and R¹ for each compound corresponds to one row of table A

Table 60

Compounds of the formula IA in which Lₙ is 2-trifluoromethyl-5-chloro, R³ is methylthio and R¹ for each compound corresponds to one row of table A

Table 61

Compounds of the formula IA in which Lₙ is 2-fluoro-6-chloro, R³ is methylsulfinyl (CH₃—S(═O)—), and R¹ for each compound corresponds to one row of table A

Table 62

Compounds of the formula IA in which Lₙ is 2,6-difluoro, R³ is methylsulfinyl, and R¹ for each compound corresponds to one row of table A

Table 63

Compounds of the formula IA in which Lₙ is 2,6-dichloro, R³ is methylsulfinyl, and R¹ for each compound corresponds to one row of table A

Table 64

Compounds of the formula IA in which Lₙ is 2-fluoro-6-methyl, R³ is methylsulfinyl, and R¹ for each compound corresponds to one row of table A

Table 65

Compounds of the formula IA in which Lₙ is 2,4,6-trifluoro, R³ is methylsulfinyl, and R¹ for each compound corresponds to one row of table A

Table 66

Compounds of the formula IA in which Lₙ is 2,6-difluoro-4-SH, R³ is methylsulfinyl, and R¹ for each compound corresponds to one row of table A

Table 67

Compounds of the formula IA in which Lₙ is pentafluoro, R³ is methylsulfinyl, and R¹ for each compound corresponds to one row of table A

Table 68

Compounds of the formula IA in which Lₙ is 2-methyl-4-fluoro, R³ is methylsulfinyl, and R¹ for each compound corresponds to one row of table A

Table 69

Compounds of the formula IA in which Lₙ is 2-trifluoromethyl, R³ is methylsulfinyl, and R¹ for each compound corresponds to one row of table A

Table 70

Compounds of the formula IA in which Lₙ is 2-methylsulfenyl-6-fluoro, R³ is methylsulfinyl, and R¹ for each compound corresponds to one row of table A

Table 71

Compounds of the formula IA in which Lₙ is 2-chloro, R³ is methylsulfinyl, and R¹ for each compound corresponds to one row of table A

Table 72

Compounds of the formula IA in which Lₙ is 2-fluoro, R³ is methylsulfinyl, and R¹ for each compound corresponds to one row of table A

Table 73

Compounds of the formula IA in which Lₙ is 2,4-difluoro, R³ is methylsulfinyl, and R¹ for each compound corresponds to one row of table A

Table 74

Compounds of the formula IA in which Lₙ is 2-fluoro-4-chloro, R³ is methylsulfinyl, and R¹ for each compound corresponds to one row of table A

Table 75

Compounds of the formula IA in which Lₙ is 2-chloro-4-fluoro, R³ is methylsulfinyl, and R¹ for each compound corresponds to one row of table A

Table 76

Compounds of the formula IA in which Lₙ is 2,3-difluoro, R³ is methylsulfinyl, and R¹ for each compound corresponds to one row of table A

Table 77

Compounds of the formula IA in which Lₙ is 2,5-difluoro, R³ is methylsulfinyl, and R¹ for each compound corresponds to one row of table A

Table 78

Compounds of the formula IA in which Lₙ is 2,3,4-trifluoro, R³ is methylsulfinyl, and R¹ for each compound corresponds to one row of table A

Table 79

Compounds of the formula IA in which Lₙ is 2-methyl, R³ is methylsulfinyl, and R¹ for each compound corresponds to one row of table A

Table 80

Compounds of the formula IA in which Lₙ is 2,4-dimethyl, R³ is methylsulfinyl, and R¹ for each compound corresponds to one row of table A

Table 81

Compounds of the formula IA in which Lₙ is 2-methyl-4-chloro, R³ is methylsulfinyl, and R¹ for each compound corresponds to one row of table A

Table 82

Compounds of the formula IA in which Lₙ is 2-fluoro-4-methyl, R³ is methylsulfinyl, and R¹ for each compound corresponds to one row of table A

Table 83

Compounds of the formula IA in which Lₙ is 2,6-dimethyl, R³ is methylsulfinyl, and R¹ for each compound corresponds to one row of table A

Table 84

Compounds of the formula IA in which Lₙ is 2,4,6-trimethyl, R³ is methylsulfinyl, and R¹ for each compound corresponds to one row of table A

Table 85

Compounds of the formula IA in which Lₙ is 2,6-difluoro-4-cyano, R³ is methylsulfinyl, and R¹ for each compound corresponds to one row of table A

Table 86

Compounds of the formula IA in which Lₙ is 2,6-difluoro-4-methyl, R³ is methylsulfinyl, and R¹ for each compound corresponds to one row of table A

Table 87

Compounds of the formula IA in which Lₙ is 2,6-difluoro-4-methoxycarbonyl, R³ is methylsulfinyl, and R¹ for each compound corresponds to one row of table A

Table 88

Compounds of the formula IA in which Lₙ is 2-trifluoromethyl-4-fluoro, R³ is methylsulfinyl, and R¹ for each compound corresponds to one row of table A

Table 89

Compounds of the formula IA in which Lₙ is 2-trifluoromethyl-5-fluoro, R³ is methylsulfinyl, and R¹ for each compound corresponds to one row of table A

Table 90

Compounds of the formula IA in which Lₙ is 2-trifluoromethyl-5-chloro, R³ is methylsulfinyl, and R¹ for each compound corresponds to one row of table A

Table 91

Compounds of the formula IA in which Lₙ is 2-fluoro-6-chloro, R³ is methylsulfonyl (CH₃—S(═O)₂—), and R¹ for each compound corresponds to one row of table A

Table 92

Compounds of the formula IA in which Lₙ is 2,6-difluoro, R³ is methylsulfonyl, and R¹ for each compound corresponds to one row of table A

Table 93

Compounds of the formula IA in which Lₙ is 2,6-dichloro, R³ is methylsulfonyl, and R¹ for each compound corresponds to one row of table A

Table 94

Compounds of the formula IA in which Lₙ is 2-fluoro-6-methyl, R³ is methylsulfonyl, and R¹ for each compound corresponds to one row of table A

Table 95

Compounds of the formula IA in which Lₙ is 2,4,6-trifluoro, R³ is methylsulfonyl, and R¹ for each compound corresponds to one row of table A

Table 96

Compounds of the formula IA in which Lₙ is 2,6-difluoro-4-SH, R³ is methylsulfonyl, and R¹ for each compound corresponds to one row of table A

Table 97

Compounds of the formula IA in which Lₙ is pentafluoro, R³ is methylsulfonyl, and R¹ for each compound corresponds to one row of table A

Table 98

Compounds of the formula IA in which Lₙ is 2-methyl-4-fluoro, R³ is methylsulfonyl, and R¹ for each compound corresponds to one row of table A

Table 99

Compounds of the formula IA in which Lₙ is 2-trifluoromethyl, R³ is methylsulfonyl, and R¹ for each compound corresponds to one row of table A

Table 100

Compounds of the formula IA in which Lₙ is 2-methylsulfenyl-6-fluoro, R³ is methylsulfonyl, and R¹ for each compound corresponds to one row of table A

Table 101

Compounds of the formula IA in which Lₙ is 2-chloro, R³ is methylsulfonyl, and R¹ for each compound corresponds to one row of table A

Table 102

Compounds of the formula IA in which Lₙ is 2-fluoro, R³ is methylsulfonyl, and R¹ for each compound corresponds to one row of table A

Table 103

Compounds of the formula IA in which Lₙ is 2,4-difluoro, R³ is methylsulfonyl, and R¹ for each compound corresponds to one row of table A

Table 104

Compounds of the formula IA in which Lₙ is 2-fluoro-4-chloro, R³ is methylsulfonyl, and R¹ for each compound corresponds to one row of table A

Table 105

Compounds of the formula IA in which Lₙ is 2-chloro-4-fluoro, R³ is methylsulfonyl, and R¹ for each compound corresponds to one row of table A

Table 106

Compounds of the formula IA in which Lₙ is 2,3-difluoro, R³ is methylsulfonyl, and R¹ for each compound corresponds to one row of table A

Table 107

Compounds of the formula IA in which Lₙ is 2,5-difluoro, R³ is methylsulfonyl, and R¹ for each compound corresponds to one row of table A

Table 108

Compounds of the formula IA in which Lₙ is 2,3,4-trifluoro, R³ is methylsulfonyl, and R¹ for each compound corresponds to one row of table A

Table 109

Compounds of the formula IA in which Lₙ is 2-methyl, R³ is methylsulfonyl, and R¹ for each compound corresponds to one row of table A

Table 110

Compounds of the formula IA in which Lₙ is 2,4-dimethyl, R³ is methylsulfonyl, and R¹ for each compound corresponds to one row of table A

Table 111

Compounds of the formula IA in which Lₙ is 2-methyl-4-chloro, R³ is methylsulfonyl, and R¹ for each compound corresponds to one row of table A

Table 112

Compounds of the formula IA in which Lₙ is 2-fluoro-4-methyl, R³ is methylsulfonyl, and R¹ for each compound corresponds to one row of table A

Table 113

Compounds of the formula IA in which Lₙ is 2,6-dimethyl, R³ is methylsulfonyl, and R¹ for each compound corresponds to one row of table A

Table 114

Compounds of the formula IA in which Lₙ is 2,4,6-trimethyl, R³ is ethylsulfonyl, and R¹ for each compound corresponds to one row of table A

Table 115

Compounds of the formula IA in which Lₙ is 2,6-difluoro-4-cyano, R³ is methylsulfonyl, and R¹ for each compound corresponds to one row of table A

Table 116

Compounds of the formula IA in which Lₙ is 2,6-difluoro-4-methyl, R³ is methylsulfonyl, and R¹ for each compound corresponds to one row of table A

Table 117

Compounds of the formula IA in which Lₙ is 2,6-difluoro-4-methoxycarbonyl, R³ is methylsulfonyl, and R¹ for each compound corresponds to one row of table A

Table 118

Compounds of the formula IA in which Lₙ is 2-trifluoromethyl-4-fluoro, R³ is methylsulfonyl, and R¹ for each compound corresponds to one row of table A

Table 119

Compounds of the formula IA in which Lₙ is 2-trifluoromethyl-5-fluoro, R³ is methylsulfonyl, and R¹ for each compound corresponds to one row of table A

Table 120

Compounds of the formula IA in which Lₙ is 2-trifluoromethyl-5-chloro, R³ is methylsulfonyl, and R¹ for each compound corresponds to one row of table A

Table 121

Compounds of the formula IA in which Lₙ is 2-fluoro-6-chloro, R³ is trifluoromethyl, and R¹ for each compound corresponds to one row of table A

Table 122

Compounds of the formula IA in which Lₙ is 2,6-difluoro, R³ is trifluoromethyl, and R¹ for each compound corresponds to one row of table A

Table 123

Compounds of the formula IA in which Lₙ is 2,6-dichloro, R³ is trifluoromethyl, and R¹ for each compound corresponds to one row of table A

Table 124

Compounds of the formula IA in which Lₙ is 2-fluoro-6-methyl, R³ is trifluoromethyl, and R¹ for each compound corresponds to one row of table A

Table 125

Compounds of the formula IA in which Lₙ is 2,4,6-trifluoro, R³ is trifluoromethyl, and R¹ for each compound corresponds to one row of table A

Table 126

Compounds of the formula IA in which Lₙ is 2,6-difluoro-4-SH, R³ is trifluoromethyl, and R¹ for each compound corresponds to one row of table A

Table 127

Compounds of the formula IA in which Lₙ is pentafluoro, R³ is trifluoromethyl, and R¹ for each compound corresponds to one row of table A

Table 128

Compounds of the formula IA in which Lₙ is 2-methyl-4-fluoro, R³ is trifluoromethyl, and R¹ for each compound corresponds to one row of table A

Table 129

Compounds of the formula IA in which Lₙ is 2-trifluoromethyl, R³ is trifluoromethyl, and R¹ for each compound corresponds to one row of table A

Table 130

Compounds of the formula IA in which Lₙ is 2-methylthio-6-fluoro, R³ is trifluoromethyl, and R¹ for each compound corresponds to one row of table A

Table 131

Compounds of the formula IA in which Lₙ is 2-chloro, R³ is trifluoromethyl, and R¹ for each compound corresponds to one row of table A

Table 132

Compounds of the formula IA in which Lₙ is 2-fluoro, R³ is trifluoromethyl, and R¹ for each compound corresponds to one row of table A

Table 133

Compounds of the formula IA in which Lₙ is 2,4-difluoro, R³ is trifluoromethyl, and R¹ for each compound corresponds to one row of table A

Table 134

Compounds of the formula IA in which Lₙ is 2-fluoro-4-chloro, R³ is trifluoromethyl, and R¹ for each compound corresponds to one row of table A

Table 135

Compounds of the formula IA in which Lₙ is 2-chloro-4-fluoro, R³ is trifluoromethyl, and R¹ for each compound corresponds to one row of table A

Table 136

Compounds of the formula IA in which Lₙ is 2,3-difluoro, R³ is trifluoromethyl, and R¹ for each compound corresponds to one row of table A

Table 137

Compounds of the formula IA in which Lₙ is 2,5-difluoro, R³ is trifluoromethyl, and R¹ for each compound corresponds to one row of table A

Table 138

Compounds of the formula IA in which Lₙ is 2,3,4-trifluoro, R³ is trifluoromethyl, and R¹ for each compound corresponds to one row of table A

Table 139

Compounds of the formula IA in which Lₙ is 2-methyl, R³ is trifluoromethyl, and R¹ for each compound corresponds to one row of table A

Table 140

Compounds of the formula IA in which Lₙ is 2,4-dimethyl, R³ is trifluoromethyl, and R¹ for each compound corresponds to one row of table A

Table 141

Compounds of the formula IA in which Lₙ is 2-methyl-4-chloro, R³ is trifluoromethyl, and R¹ for each compound corresponds to one row of table A

Table 142

Compounds of the formula IA in which Lₙ is 2-fluoro-4-methyl, R³ is trifluoromethyl, and R¹ for each compound corresponds to one row of table A

Table 143

Compounds of the formula IA in which Lₙ is 2,6-dimethyl, R³ is trifluoromethyl, and R¹ for each compound corresponds to one row of table A

Table 144

Compounds of the formula IA in which Lₙ is 2,4,6-trimethyl, R³ is trifluoromethyl, and R¹ for each compound corresponds to one row of table A

Table 145

Compounds of the formula IA in which Lₙ is 2,6-difluoro-4-cyano, R³ is trifluoromethyl, and R¹ for each compound corresponds to one row of table A

Table 146

Compounds of the formula IA in which Lₙ is 2,6-difluoro-4-methyl, R³ is trifluoromethyl, and R¹ for each compound corresponds to one row of table A

Table 147

Compounds of the formula IA in which Lₙ is 2,6-difluoro-4-methoxycarbonyl, R³ is trifluoromethyl, and R¹ for each compound corresponds to one row of table A

Table 148

Compounds of the formula IA in which Lₙ is 2-trifluoromethyl-4-fluoro, R³ is trifluoromethyl, and R¹ for each compound corresponds to one row of table A

Table 149

Compounds of the formula IA in which Lₙ is 2-trifluoromethyl-5-fluoro, R³ is trifluoromethyl, and R¹ for each compound corresponds to one row of table A

Table 150

Compounds of the formula IA in which Lₙ is 2-trifluoromethyl-5-chloro, R³ is trifluoromethyl, and R¹ for each compound corresponds to one row of table A

TABLE A

| No. | R¹ |
|---|---|
| A-1 | CH₃ |
| A-2 | CH₂CH₃ |
| A-3 | CH₂CH₂CH₃ |
| A-4 | CH(CH₃)₂ |
| A-5 | CH₂CH(CH₃)₂ |
| A-6 | (±) CH(CH₃)CH₂CH₃ |
| A-7 | (R) CH(CH₃)CH₂CH₃ |
| A-8 | (S) CH(CH₃)CH₂CH₃ |
| A-9 | (CH₂)₃CH₃ |
| A-10 | C(CH₃)₃ |
| A-11 | (CH₂)₄CH₃ |
| A-12 | CH(CH₂CH₃)₂ |
| A-13 | CH₂CH₂CH(CH₃)₂ |
| A-14 | (±) CH(CH₃)(CH₂)₂CH₃ |
| A-15 | (R) CH(CH₃)(CH₂)₂CH₃ |
| A-16 | (S) CH(CH₃)(CH₂)₂CH₃ |
| A-17 | (±) CH₂CH(CH₃)CH₂CH₃ |
| A-18 | (R) CH₂CH(CH₃)CH₂CH₃ |
| A-19 | (S) CH₂CH(CH₃)CH₂CH₃ |
| A-20 | (±) CH(CH₃)CH(CH₃)₂ |
| A-21 | (R) CH(CH₃)CH(CH₃)₂ |
| A-22 | (S) CH(CH₃)CH(CH₃)₂ |
| A-23 | (CH₂)₅CH₃ |
| A-24 | (±, ±) CH(CH₃)CH(CH₃)CH₂CH₃ |
| A-25 | (±, R) CH(CH₃)CH(CH₃)CH₂CH₃ |
| A-26 | (±, S) CH(CH₃)CH(CH₃)CH₂CH₃ |
| A-27 | (±) CH₂CH(CH₃)CF₃ |
| A-28 | (R) CH₂CH(CH₃)CF₃ |
| A-29 | (S) CH₂CH(CH₃)CF₃ |
| A-30 | (±) CH₂CH(CF₃)CH₂CH₃ |
| A-31 | (R) CH₂CH(CF₃)CH₂CH₃ |
| A-32 | (S) CH₂CH(CF₃)CH₂CH₃ |
| A-33 | (±, ±) CH(CH₃)CH(CH₃)CF₃ |
| A-34 | (±, R) CH(CH₃)CH(CH₃)CF₃ |
| A-35 | (±, S) CH(CH₃)CH(CH₃)CF₃ |
| A-36 | (±, ±) CH(CH₃)CH(CF₃)CH₂CH₃ |
| A-37 | (±, R) CH(CH₃)CH(CF₃)CH₂CH₃ |
| A-38 | (±, S) CH(CH₃)CH(CF₃)CH₂CH₃ |
| A-39 | CF₃ |
| A-40 | CF₂CF₃ |
| A-41 | CF₂CF₂CF₃ |
| A-42 | c-C₃H₅ |
| A-43 | (1-CH₃)-c-C₃H₄ |
| A-44 | c-C₅H₉ |
| A-45 | c-C₆H₁₁ |
| A-46 | (4-CH₃)-c-C₆H₁₀ |
| A-47 | CH₂C(CH₃)═CH₂ |
| A-48 | CH₂CH₂C(CH₃)═CH₂ |
| A-49 | CH₂—C(CH₃)₃ |
| A-50 | CH₂—Si(CH₃)₃ |
| A-51 | n-C₆H₁₃ |
| A-52 | (CH₂)₃—CH(CH₃)₂ |
| A-53 | (CH₂)₂—CH(CH₃)—C₂H₅ |
| A-54 | CH₂—CH(CH₃)-n-C₃H₇ |
| A-55 | CH(CH₃)-n-C₄H₉ |
| A-56 | CH₂—CH(C₂H₅)₂ |
| A-57 | CH(C₂H₅)-n-C₃H₇ |
| A-58 | CH₂-c-C₅H₉ |
| A-59 | CH₂—CH(CH₃)—CH(CH₃)₂ |
| A-60 | CH(CH₃)—CH₂CH(CH₃)₂ |
| A-61 | CH(CH₃)—CH(CH₃)—C₂H₅ |
| A-62 | CH(CH₃)—C(CH₃)₃ |
| A-63 | (CH₂)₂—C(CH₃)₃ |
| A-64 | CH₂—C(CH₃)₂—C₂H₅ |
| A-65 | 2-CH₃-c-C₅H₈ |
| A-66 | 3-CH₃-c-C₅H₈ |
| A-67 | C(CH₃)₂-n-C₃H₇ |
| A-68 | (CH₂)₆—CH₃ |
| A-69 | (CH₂)₄—CH(CH₃)₂ |
| A-70 | (CH₂)₃—CH(CH₃)—C₂H₅ |
| A-71 | (CH₂)₂—CH(CH₃)-n-C₃H₇ |
| A-72 | CH₂—CH(CH₃)-n-C₄H₉ |
| A-73 | CH(CH₃)-n-C₅H₁₁ |
| A-74 | (CH₂)₃C(CH₃)₃ |
| A-75 | (CH₂)₂CH(CH₃)—CH(CH₃)₂ |
| A-76 | (CH₂)CH(CH₃)—CH₂CH(CH₃)₂ |
| A-77 | CH(CH₃)(CH₂)₂—CH(CH₃)₂ |
| A-78 | (CH₂)₂C(CH₃)₂C₂H₅ |
| A-79 | CH₂CH(CH₃)CH(CH₃)C₂H₅ |
| A-80 | CH(CH₃)CH₂CH(CH₃)C₂H₅ |
| A-81 | CH₂C(CH₃)₂-n-C₃H₇ |
| A-82 | CH(CH₃)CH(CH₃)-n-C₃H₇ |
| A-83 | C(CH₃)₂-n-C₄H₉ |
| A-84 | (CH₂)₂CH(C₂H₅)₂ |
| A-85 | CH₂CH(C₂H₅)-n-C₃H₇ |
| A-86 | CH(C₂H₅)-n-C₄H₉ |
| A-87 | CH₂CH(CH₃)C(CH₃)₃ |
| A-88 | CH(CH₃)CH₂C(CH₃)₃ |
| A-89 | CH₂C(CH₃)₂CH(CH₃)₂ |
| A-90 | CH₂CH(C₂H₅)CH(CH₃)₂ |
| A-91 | CH(CH₃)CH(CH₃)CH(CH₃)₂ |
| A-92 | C(CH₃)₂CH₂CH(CH₃)₂ |
| A-93 | CH(C₂H₅)CH₂CH(CH₃)₂ |
| A-94 | CH(CH₃)C(CH₃)₂C₂H₅ |
| A-95 | CH(CH₃)CH(C₂H₅)₂ |
| A-96 | C(CH₃)₂CH(CH₃)C₂H₅ |
| A-97 | CH(C₂H₅)CH(CH₃)C₂H₅ |
| A-98 | C(CH₃)(C₂H₅)-n-C₃H₇ |
| A-99 | CH(n-C₃H₇)₂ |
| A-100 | CH(n-C₃H₇)CH(CH₃)₂ |
| A-101 | C(CH₃)₂C(CH₃)₃ |
| A-102 | C(CH₃)(C₂H₅)—CH(CH₃)₂ |
| A-103 | C(C₂H₅)₃ |
| A-104 | (3-CH₃)-c-C₆H₁₀ |
| A-105 | (2-CH₃)-c-C₆H₁₀ |
| A-106 | n-C₈H₁₇ |
| A-107 | CH₂C(═NO—CH₃)CH₃ |
| A-108 | CH₂C(═NO—C₂H₅)CH₃ |
| A-109 | CH₂C(═NO-n-C₃H₇)CH₃ |
| A-110 | CH₂C(═NO-i-C₃H₇)CH₃ |
| A-111 | CH(CH₃)C(═NOCH₃)CH₃ |
| A-112 | CH(CH₃)C(═NOC₂H₅)CH₃ |
| A-113 | CH(CH₃)C(═NO-n-C₃H₇)CH₃ |
| A-114 | CH(CH₃)C(═NO-i-C₃H₇)CH₃ |
| A-115 | C(═NOCH₃)C(═NOCH₃)CH₃ |
| A-116 | C(═NOCH₃)C(═NOC₂H₅)CH₃ |
| A-117 | C(═NOCH₃)C(═NO-n-C₃H₇)CH₃ |
| A-118 | C(═NOCH₃)C(═NO-i-C₃H₇)CH₃ |
| A-119 | C(═NOC₂H₅)C(═NOCH₃)CH₃ |
| A-120 | C(═NOC₂H₅)C(═NOC₂H₅)CH₃ |
| A-121 | C(═NOC₂H₅)C(═NO-n-C₃H₇)CH₃ |
| A-122 | C(═NOC₂H₅)C(═NO-i-C₃H₇) CH₃ |
| A-123 | CH₂C(═NO—CH₃)C₂H₅ |
| A-124 | CH₂C(═NO—C₂H₅)C₂H₅ |
| A-125 | CH₂C(═NO-n-C₃H₇)C₂H₅ |
| A-126 | CH₂C(═NO-i-C₃H₇)C₂H₅ |
| A-127 | CH(CH₃)C(═NOCH₃)C₂H₅ |
| A-128 | CH(CH₃)C(═NOC₂H₅)C₂H₅ |
| A-129 | CH(CH₃)C(═NO-n-C₃H₇)C₂H₅ |
| A-130 | CH(CH₃)C(═NO-n-C₃H₇)C₂H₅ |
| A-131 | C(═NOCH₃)C(═NOCH₃)C₂H₅ |
| A-132 | C(═NOCH₃)C(═NOC₂H₅)C₂H₅ |
| A-133 | C(═NOCH₃)C(═NO-n-C₃H₇)C₂H₅ |
| A-134 | C(═NOCH₃)C(═NO-i-C₃H₇)C₂H₅ |
| A-135 | C(═NOC₂H₅)C(═NOCH₃)C₂H₅ |
| A-136 | C(═NOC₂H₅)C(═NOC₂H₅)C₂H₅ |
| A-137 | C(═NOC₂H₅)C(═NO-n-C₃H₇)C₂H₅ |
| A-138 | C(═NOC₂H₅)C(═NO-i-C₃H₇)C₂H₅ |
| A-139 | CH═CH—CH₂CH₃ |
| A-140 | CH₂—CH═CH—CH₃ |
| A-141 | CH₂—CH₂—CH═CH₂ |
| A-142 | C(CH₃)₂CH₂CH₃ |
| A-143 | CH═C(CH₃)₂ |
| A-144 | C(═CH₂)—CH₂CH₃ |
| A-145 | C(CH₃)═CH—CH₃ |
| A-146 | CH(CH₃)CH═CH₂ |
| A-147 | CH═CH-n-C₃H₇ |
| A-148 | CH₂—CH═CH—C₂H₅ |
| A-149 | (CH₂)₂—CH═CH—CH₃ |
| A-150 | (CH₂)₃—CH═CH₂ |
| A-151 | CH═CH—CH(CH₃)₂ |
| A-152 | CH₂—CH═C(CH₃)₂ |
| A-153 | (CH₂)₂—C(CH₃)═CH₂ |
| A-154 | CH═C(CH₃)—C₂H₅ |
| A-155 | CH₂—C(═CH₂)—C₂H₅ |
| A-156 | CH₂—C(CH₃)═CH—CH₃ |
| A-157 | CH₂—CH(CH₃)—CH═CH₂ |
| A-158 | C(═CH₂)—CH₂—CH₂—CH₃ |
| A-159 | C(CH₃)═CH—CH₂—CH₃ |
| A-160 | CH(CH₃)—CH═CH—CH₃ |
| A-161 | CH(CH₃)—CH₂—CH═CH₂ |
| A-162 | C(═CH₂)CH(CH₃)₂ |
| A-163 | C(CH₃)═C(CH₃)₂ |
| A-164 | CH(CH₃)—C(═CH₂)—CH₃ |
| A-165 | C(CH₃)₂—CH═CH₂ |
| A-166 | C(C₂H₅)═CH—CH₃ |
| A-167 | CH(C₂H₅)—CH═CH₂ |
| A-168 | CH═CH—CH₂—CH₂—CH₂—CH₃ |
| A-169 | CH₂—CH═CH—CH₂—CH₂—CH₃ |
| A-170 | CH₂—CH₂—CH═CH—CH₂—CH₃ |
| A-171 | CH₂—CH₂—CH₂—CH═CH—CH₃ |
| A-172 | CH₂—CH₂—CH₂—CH₂—CH═CH₂ |
| A-173 | CH═CH—CH₂—CH(CH₃)CH₃ |
| A-174 | CH₂—CH═CH—CH(CH₃)CH₃ |
| A-175 | CH₂—CH₂—CH═C(CH₃)CH₃ |
| A-176 | CH₂—CH₂—CH₂—C(CH₃)═CH₂ |
| A-177 | CH═CH—CH(CH₃)—CH₂—CH₃ |
| A-178 | CH₂—CH═C(CH₃)—CH₂—CH₃ |
| A-179 | CH₂—CH₂—C(═CH₂)—CH₂—CH₃ |
| A-180 | CH₂—CH₂—C(CH₃)CH—CH₃ |
| A-181 | CH₂—CH₂—CH(CH₃)—CH═CH₂ |
| A-182 | CH═C(CH₃)—CH₂—CH₂—CH₃ |
| A-183 | CH₂—C(═CH₂)—CH₂—CH₂—CH₃ |
| A-184 | CH₂—C(CH₃)═CH—CH₂—CH₃ |
| A-185 | CH₂—CH(CH₃)—CH═CH—CH₃ |
| A-186 | CH₂—CH(CH₃)—CH₂—CH═CH₂ |
| A-187 | C(═CH₂)—CH₂—CH₂—CH₂—CH₃ |
| A-188 | C(CH₃)═CH—CH₂—CH₂—CH₃ |
| A-189 | CH(CH₃)—CH═CH—CH₂—CH₃ |
| A-190 | CH(CH₃)—CH₂—CH═CH—CH₃ |
| A-191 | CH(CH₃)—CH₂—CH₂—CH═CH₂ |
| A-192 | CH═CH—C(CH₃)₃ |
| A-193 | CH═C(CH₃)—CH(CH₃)—CH₃ |
| A-194 | CH₂—C(═CH₂)—CH(CH₃)—CH₃ |
| A-195 | CH₂—C(CH₃)═C(CH₃)—CH₃ |
| A-196 | CH₂—CH(CH₃)—C(═CH₂)—CH₃ |
| A-197 | C(═CH₂)—CH₂—CH(CH₃)—CH₃ |
| A-198 | C(CH₃)═CH—CH(CH₃)—CH₃ |
| A-199 | CH(CH₃)—CH═C(CH₃)—CH₃ |
| A-200 | CH(CH₃)—CH₂—C(═CH₂)—CH₃ |
| A-201 | CH═C(CH₂—CH₃)—CH₂—CH₃ |
| A-202 | CH₂—C(═CH—CH₃)—CH₂—CH₃ |
| A-203 | CH₂—CH(CH═CH₂)—CH₂—CH₃ |
| A-204 | C(═CH—CH₃)—CH₂—CH₂—CH₃ |
| A-205 | CH(CH═CH₂)—CH₂—CH₂—CH₃ |
| A-206 | C(CH₂—CH₃)═CH—CH₂—CH₃ |
| A-207 | CH(CH₂—CH₃)—CH═CH—CH₃ |
| A-208 | CH(CH₂—CH₃)—CH₂—CH═CH₂ |
| A-209 | CH₂—C(CH₃)₂—CH═CH₂ |
| A-210 | C(═CH₂)—CH(CH₃)—CH₂—CH₃ |
| A-211 | C(CH₃)═C(CH₃)—CH₂—CH₃ |
| A-212 | CH(CH₃)—C(═CH₂)—CH₂—CH₃ |
| A-213 | CH(CH₃)—C(CH₃)═CH—CH₃ |
| A-214 | CH(CH₃)—CH(CH₃)—CH═CH₂ |
| A-215 | C(CH₃)₂—CH═CH—CH₃ |
| A-216 | C(CH₃)₂—CH₂—CH═CH₂ |
| A-217 | C(═CH₂)—C(CH₃)₃ |
| A-218 | C(═CH—CH₃)—CH(CH₃)—CH₃ |
| A-219 | CH(CH═CH₂)—CH(CH₃)—CH₃ |
| A-220 | C(CH₂—CH₃)═C(CH₃)—CH₃ |
| A-221 | CH(CH₂—CH₃)—C(═CH₂)—CH₃ |
| A-222 | C(CH₃)₂—C(═CH₂)—CH₃ |
| A-223 | C(CH₃)(CH═CH₂)—CH₂—CH₃ |
| A-224 | C(CH₃)(CH₂CH₃)—CH₂—CH₂—CH₃ |
| A-225 | CH(CH₂CH₃)—CH(CH₃)—CH₂—CH₃ |
| A-226 | CH(CH₂CH₃)—CH₂—CH(CH₃)—CH₃ |
| A-227 | C(CH₃)₂—C(CH₃)₃ |
| A-228 | C(CH₂—CH₃)—C(CH₃)₃ |
| A-229 | C(CH₃)(CH₂—CH₃)—CH(CH₃)₂ |
| A-230 | CH(CH(CH₃)₂)—CH(CH₃)₂ |
| A-231 | CH═CH—CH₂—CH₂—CH₂—CH₂—CH₃ |
| A-232 | CH₂—CH═CH—CH₂—CH₂—CH₂—CH₃ |
| A-233 | CH₂—CH₂—CH═CH—CH₂—CH₂—CH₃ |
| A-234 | CH₂—CH₂—CH₂—CH═CH—CH₂—CH₃ |
| A-235 | CH₂—CH₂—CH₂—CH₂—CH═CH—CH₃ |
| A-236 | CH₂—CH₂—CH₂—CH₂—CH₂—CH═CH₂ |
| A-237 | CH═CH—CH₂—CH₂—CH(CH₃)—CH₃ |
| A-238 | CH₂—CH═CH—CH₂—CH(CH₃)—CH₃ |
| A-239 | CH₂—CH₂—CH═CH—CH(CH₃)—CH₃ |
| A-240 | CH₂—CH₂—CH₂—CH═C(CH₃)—CH₃ |
| A-241 | CH₂—CH₂—CH₂—CH₂—C(═CH₂)—CH₃ |
| A-242 | CH═CH—CH₂—CH(CH₃)—CH₂—CH₃ |
| A-243 | CH₂—CH═CH—CH(CH₃)—CH₂—CH₃ |
| A-244 | CH₂—CH₂—CH═C(CH₃)—CH₂—CH₃ |
| A-245 | CH₂—CH₂—CH₂—C(═CH₂)—CH₂—CH₃ |
| A-246 | CH₂—CH₂—CH₂—C(CH₃)═CH—CH₃ |
| A-247 | CH₂—CH₂—CH₂—CH(CH₃)—CH═CH₂ |
| A-248 | CH═CH—CH(CH₃)—CH₂—CH₂—CH₃ |
| A-249 | CH₂—CH═C(CH₃)—CH₂—CH₂—CH₃ |
| A-250 | CH₂—CH₂—C(═CH₂)—CH₂—CH₂—CH₃ |
| A-251 | CH₂—CH₂—C(CH₃)═CH—CH₂—CH₃ |
| A-252 | CH₂—CH₂—CH(CH₃)—CH═CH—CH₃ |
| A-253 | CH₂—CH₂—CH(CH₃)—CH₂—CH═CH₂ |
| A-254 | CH═C(CH₃)—CH₂—CH₂—CH₂—CH₃ |
| A-255 | CH₂—C(═CH₂)—CH₂—CH₂—CH₂—CH₃ |
| A-256 | CH₂—C(CH₃)═CH—CH₂—CH₂—CH₃ |
| A-257 | CH₂—CH(CH₃)—CH═CH—CH₂—CH₃ |
| A-258 | CH₂—CH(CH₃)—CH₂—CH═CH—CH₃ |
| A-259 | CH₂—CH(CH₃)—CH₂—CH₂—CH═CH₂ |
| A-260 | C(═CH₂)—CH₂—CH₂—CH₂—CH₂—CH₃ |
| A-261 | C(CH₃)═CH—CH₂—CH₂—CH₂—CH₃ |
| A-262 | CH(CH₃)—CH═CH—CH₂—CH₂—CH₃ |
| A-263 | CH(CH₃)—CH₂—CH═CH—CH₂—CH₃ |
| A-264 | CH(CH₃)—CH₂—CH₂—CH═CH—CH₃ |
| A-265 | CH(CH₃)—CH₂—CH₂—CH₂—CH═CH₂ |
| A-266 | CH═CH—CH₂—C(CH₃)₃ |
| A-267 | CH₂—CH═CH—C(CH₃)₃ |
| A-268 | CH═CH—CH(CH₃)—CH(CH₃)₂ |
| A-269 | CH₂—CH═C(CH₃)—CH(CH₃)₂ |
| A-270 | CH₂—CH₂—C(═CH₂)—CH(CH₃)₂ |
| A-271 | CH₂—CH₂—C(CH₃)═C(CH₃)₂ |
| A-272 | CH₂—CH₂—CH(CH₃)—C(═CH₂)—CH₃ |
| A-273 | CH═C(CH₃)—CH₂—CH(CH₃)₂ |
| A-274 | CH₂—C(═CH₂)—CH₂—CH(CH₃)₂ |
| A-275 | CH₂—C(CH₃)═CH—CH(CH₃)₂ |
| A-276 | CH₂—CH(CH₃)—CH═C(CH₃)₂ |
| A-277 | CH₂—CH(CH₃)—CH₂—C(═CH₂)—CH₃ |
| A-278 | C(═CH₂)—CH₂—CH₂—CH(CH₃)₂ |
| A-279 | C(CH₃)═CH—CH₂—CH(CH₃)₂ |
| A-280 | CH(CH₃)—CH═CH—CH(CH₃)₂ |
| A-281 | CH(CH₃)—CH₂—CH═C(CH₃)₂ |
| A-282 | CH(CH₃)—CH₂—CH₂—C(═CH₂)—CH₃ |
| A-283 | CH═CH—C(CH₃)₂—CH₂—CH₃ |
| A-284 | CH₂—CH₂—C(CH₃)₂—CH═CH₂ |
| A-285 | CH═C(CH₃)—CH(CH₃)—CH₂—CH₃ |
| A-286 | CH₂—C(═CH₂)—CH(CH₃)—CH₂—CH₃ |
| A-287 | CH₂—C(CH₃)═C(CH₃)—CH₂—CH₃ |
| A-288 | CH₂—CH(CH₃)—C(═CH₂)—CH₂—CH₃ |
| A-289 | CH₂—CH(CH₃)—C(CH₃)═CH—CH₃ |
| A-290 | CH₂—CH(CH₃)—CH(CH₃)—CH═CH₂ |
| A-291 | C(═CH₂)—CH₂—CH(CH₃)—CH₂—CH₃ |
| A-292 | C(CH₃)═CH—CH(CH₃)—CH₂—CH₃ |
| A-293 | CH(CH₃)—CH═C(CH₃)—CH₂—CH₃ |
| A-294 | CH(CH₃)—CH₂—C(═CH₂)—CH₂—CH₃ |
| A-295 | CH(CH₃)—CH₂—C(CH₃)═CH—CH₃ |
| A-296 | CH(CH₃)—CH₂—CH(CH₃)—CH═CH₂ |
| A-297 | CH₂—C(CH₃)₂—CH═CH—CH₃ |
| A-298 | CH₂—C(CH₃)₂—CH₂—CH═CH₂ |
| A-299 | C(═CH₂)—CH(CH₃)—CH₂—CH₂—CH₃ |
| A-300 | C(CH₃)═C(CH₃)—CH₂—CH₂—CH₃ |
| A-301 | CH(CH₃)—C(═CH₂)—CH₂—CH₂—CH₃ |
| A-302 | CH(CH₃)—C(CH₃)═CH—CH₂—CH₃ |
| A-303 | CH(CH₃)—CH(CH₃)—CH═CH—CH₃ |
| A-304 | CH(CH₃)—CH(CH₃)—CH₂—CH═CH₂ |
| A-305 | C(CH₃)₂—CH═CH—CH₂—CH₃ |
| A-306 | C(CH₃)₂—CH₂—CH═CH—CH₃ |
| A-307 | C(CH₃)₂—CH₂—CH₂—CH═CH₂ |
| A-308 | CH═CH—CH(CH₂—CH₃)—CH₂—CH₃ |
| A-309 | CH₂—CH═C(CH₂—CH₃)—CH₂—CH₃ |
| A-310 | CH₂—CH₂—C(═CH—CH₃)—CH₂—CH₃ |
| A-311 | CH₂—CH₂—CH(CH═CH₂)—CH₂—CH₃ |
| A-312 | CH═C(CH₂—CH₃)—CH₂—CH₂—CH₃ |
| A-313 | CH₂—C(═CH—CH₃)—CH₂—CH₂—CH₃ |
| A-314 | CH₂—CH(CH═CH₂)—CH₂—CH₂—CH₃ |
| A-315 | CH₂—C(CH₂—CH₃)═CH—CH₂—CH₃ |
| A-316 | CH₂—CH(CH₂—CH₃)—CH═CH—CH₃ |
| A-317 | CH₂—CH(CH₂—CH₃)—CH—CH═CH₂ |
| A-318 | C(═CH—CH₃)—CH₂—CH₂—CH₂—CH₃ |
| A-319 | CH(CH═CH₂)—CH₂—CH₂—CH₂—CH₃ |
| A-320 | C(CH₂—CH₃)═CH—CH₂—CH₂—CH₃ |
| A-321 | CH(CH₂—CH₃)—CH═CH—CH₂—CH₃ |
| A-322 | CH(CH₂—CH₃)—CH₂—CH═CH—CH₃ |
| A-323 | CH(CH₂—CH₃)—CH₂—CH₂—CH═CH₂ |
| A-324 | C(═CH—CH₂—CH₃)—CH₂—CH₂—CH₃ |
| A-325 | C(CH═CH—CH₃)—CH₂—CH₂—CH₃ |
| A-326 | C(CH₂—CH═CH₂)—CH₂—CH₂—CH₃ |
| A-327 | CH═C(CH₃)—C(CH₃)₃ |
| A-328 | CH₂—C(═CH₂)—C(CH₃)₃ |
| A-329 | CH₂—C(CH₃)₂—CH(═CH₂)—CH₃ |
| A-330 | C(═CH₂)—CH(CH₃)—CH(CH₃)—CH₃ |
| A-331 | C(CH₃)═C(CH₃)—CH(CH₃)—CH₃ |
| A-332 | CH(CH₃)—C(═CH₂)—CH(CH₃)—CH₃ |
| A-333 | CH(CH₃)—C(CH₃)═C(CH₃)—CH₃ |
| A-334 | CH(CH₃)—CH(CH₃)—C(═CH₂)—CH₃ |
| A-335 | C(CH₃)₂—CH═C(CH₃)—CH₃ |
| A-336 | C(CH₃)₂—CH₂—C(═CH₂)—CH₃ |
| A-337 | C(CH₃)₂—C(═CH₂)—CH₂—CH₃ |
| A-338 | C(CH₃)₂—C(CH₃)═CH—CH₃ |
| A-339 | C(CH₃)₂—CH(CH₃)CH═CH₂ |
| A-340 | CH(CH₂—CH₃)—CH₂—CH(CH₃)—CH₃ |
| A-341 | CH(CH₂—CH₃)—CH(CH₃)—CH₂—CH₃ |
| A-342 | C(CH₃)(CH₂—CH₃)—CH₂—CH₂—CH₃ |
| A-343 | CH(i-C₃H₇)—CH₂—CH₂—CH₃ |
| A-344 | CH═C(CH₂—CH₃)—CH(CH₃)—CH₃ |
| A-345 | CH₂—C(═CH—CH₃)—CH(CH₃)—CH₃ |
| A-346 | CH₂—CH(CH═CH₂)—CH(CH₃)—CH₃ |
| A-347 | CH₂—C(CH₂—CH₃)═C(CH₃)—CH₃ |
| A-348 | CH₂—CH(CH₂—CH₃)—C(═CH₂)—CH₃ |
| A-349 | CH₂—C(CH₃)(CH═CH₂)—CH₂—CH₃ |
| A-350 | C(═CH₂)—CH(CH₂—CH₃)—CH₂—CH₃ |
| A-351 | C(CH₃)═C(CH₂—CH₃)—CH₂—CH₃ |
| A-352 | CH(CH₃)—C(═CH—CH₃)—CH₂—CH₃ |
| A-353 | CH(CH₃)—CH(CH═CH₂)—CH₂—CH₃ |
| A-354 | CH═C(CH₂—CH₃)—CH(CH₃)—CH₃ |
| A-355 | CH₂—C(═CH—CH₃)—CH(CH₃)—CH₃ |
| A-356 | CH₂—CH(CH═CH₂)—CH(CH₃)—CH₃ |
| A-357 | CH₂—C(CH₂—CH₃)═C(CH₃)—CH₃ |
| A-358 | CH₂—CH(CH₂—CH₃)—C(═CH₂)—CH₃ |
| A-359 | C(═CH—CH₃)—CH₂—CH(CH₃)—CH₃ |
| A-360 | CH(CH═CH₂)—CH₂—CH(CH₃)—CH₃ |
| A-361 | C(CH₂—CH₃)═CH—CH(CH₃)—CH₃ |
| A-362 | CH(CH₂—CH₃)CH═C(CH₃)—CH₃ |
| A-363 | CH(CH₂—CH₃)CH₂—C(═CH₂)—CH₃ |
| A-364 | C(═CH—CH₃)CH(CH₃)—CH₂—CH₃ |
| A-365 | CH(CH═CH₂)CH(CH₃)—CH₂—CH₃ |
| A-366 | C(CH₂—CH₃)═C(CH₃)—CH₂—CH₃ |
| A-367 | CH(CH₂—CH₃)—C(═CH₂)—CH₂—CH₃ |
| A-368 | CH(CH₂—CH₃)—C(CH₃)═CH—CH₃ |
| A-369 | CH(CH₂—CH₃)—CH(CH₃)—CH═CH₂ |
| A-370 | C(CH₃)(CH═CH₂)—CH₂—CH₂—CH₃ |
| A-371 | C(CH₃)(CH₂—CH₃)—CH═CH—CH₃ |
| A-372 | C(CH₃)(CH₂—CH₃)—CH₂—CH═CH₂ |
| A-373 | C[═C(CH₃)—CH₃]—CH₂—CH₂—CH₃ |
| A-374 | CH[C(═CH₂)—CH₃]—CH₂—CH₂—CH₃ |
| A-375 | C(i-C₃H₇)═CH—CH₂—CH₃ |
| A-376 | CH(i-C₃H₇)—CH═CH—CH₃ |
| A-377 | CH(i-C₃H₇)—CH₂—CH═CH₂ |
| A-378 | C(═CH—CH₃)—C(CH₃)₃ |
| A-379 | CH(CH═CH₂)—C(CH₃)₃ |
| A-380 | C(CH₃)(CH═CH₂)CH(CH₃)—CH₃ |
| A-381 | C(CH₃)(CH₂—CH₃)C(═CH₂)—CH₃ |
| A-382 | 2-CH₃-Cyclohex-1-enyl |
| A-383 | [2-(═CH₂)]-c-C₆H₉ |
| A-384 | 2-CH₃-Cyclohex-2-enyl |
| A-385 | 2-CH₃-Cyclohex-3-enyl |
| A-386 | 2-CH₃-Cyclohex-4-enyl |
| A-387 | 2-CH₃-Cyclohex-5-enyl |
| A-388 | 2-CH₃-Cyclohex-6-enyl |
| A-389 | 3-CH₃-Cyclohex-1-enyl |
| A-390 | 3-CH₃-Cyclohex-2-enyl |
| A-391 | [3-(═CH₂)]-c-C₆H₉ |
| A-392 | 3-CH₃-Cyclohex-3-enyl |
| A-393 | 3-CH₃-Cyclohex-4-enyl |
| A-394 | 3-CH₃-Cyclohex-5-enyl |
| A-395 | 3-CH₃-Cyclohex-6-enyl |
| A-396 | 4-CH₃-Cyclohex-1-enyl |
| A-397 | 4-CH₃-Cyclohex-2-enyl |
| A-398 | 4-CH₃-Cyclohex-3-enyl |
| A-399 | [4-(CH₂)]-c-C₆H₉ |

The compounds I are suitable as fungicides. They have excellent activity against a broad spectrum of phytopathogenic fungi, in particular from the class of the Ascomycetes, Deuteromycetes, Oomycetes and Basidiomycetes. Some of them act systemically and can be employed in crop protection as foliar- and soil-acting fungicides.

They are especially important for controlling a large number of fungi in a variety of crop plants such as wheat, rye, barley, oats, rice, maize, grass, bananas, cotton, soybean, coffee, sugar cane, grapevines, fruit species, ornamentals and vegetable species such as cucumbers, beans, tomatoes, potatoes and cucurbits, and also in the seeds of these plants.

Specifically, they are suitable for controlling the following plant diseases:
Alternaria species in vegetables and fruit,
Bipolaris and Drechslera species in cereals, rice and lawns,
Blumeria graminis (powdery mildew) in cereals,
Botrytis cinerea (gray mold) in strawberries, vegetables, ornamentals and grapevines,
Erysiphe cichoracearum and Sphaerotheca fuliginea in cucurbits,
Fusarium and Verticillium species in a variety of plants,
Mycosphaerella species in cereals, bananas and groundnuts,
Phytophthora infestans in potatoes and tomatoes,
Plasmopara viticola in grapevines,
Podosphaera leucotricha in apples,
Pseudocercosporella herpotrichoides in wheat and barley,
Pseudoperonospora species in hops and cucumbers,
Puccinia species in cereals,
Pyricularia oryzae in rice,
Rhizoctonia species in cotton, rice and lawns,
Septoria tritici and Stagonospora nodorum in wheat,
Uncinula necator in grapevines,
Ustilago species in cereals and sugar cane, and
Venturia species (scab) in apples and pears.

The compounds I are also suitable for controlling harmful fungi such as Paecilomyces variotii in the protection of materials (for example wood, paper, paint dispersions, fibers or tissues) and in the protection of stored products.

The compounds I are employed by treating the fungi or the plants, seeds, materials or the soil to be protected against fungal attack with a fungicidally effective amount of the active compounds. The application can be carried out before or after the infection of the materials, plants or seeds by the fungi.

The fungicidal compositions generally comprise from 0.1 to 95, preferably from 0.5 to 90, % by weight of active compound.

For use in crop protection, the application rates are, depending on the kind of effect desired, from 0.01 to 2.0 kg of active compound per ha.

The treatment of seeds generally requires active compound rates of from 0.001 to 0.1 g, preferably from 0.01 to 0.05 g, per kilogram of seed.

For use in the protection of materials or stored products, the active compound application rate depends on the kind of application area and effect desired. Customary application rates in the protection of materials are, for example, from 0.001 g to 2 kg, preferably from 0.005 g to 1 kg, of active compound per cubic meter of treated material.

The compounds I can be converted into the customary formulations, e.g. solutions, emulsions, suspensions, dusts, powders, pastes and granules. The use form depends on the particular intended use; in any case, it should ensure fine and uniform distribution of the compound according to the invention.

The formulations are prepared in a known manner, e.g. by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants which are used are the alkali metal, alkaline earth metal and ammonium salts of lignosulfonic acid, naphthalenesulfonic acid, phenolsulfonic acid, and dibutylnaphthalenesulfonic acid, alkylarylsulfonates, alkyl sulfates, alkylsulfonates, fatty alcohol sulfates, fatty acids and sulfated fatty alcohol glycol ethers, furthermore condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol, nonylphenol, alkylphenol polyglycol ethers, tributylphenyl polyglycol ethers, tristerylphenyl polyglycol ethers, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose are suitable.

Suitable for preparing directly sprayable solutions, emulsions, pastes or oil dispersions are petroleum fractions having medium to high boiling points, such as kerosene or diesel oil, furthermore coal-tar oils and oils of plant or animal origin, aliphatic, cyclic and aromatic hydrocarbons, for example toluene, xylene, paraffin, tetrahydronaphthalene, alkylated naphthalenes or derivatives thereof, methanol, ethanol, propanol, butanol, cyclohexanol, cyclohexanone, isophorone, strongly polar solvents, for example dimethyl sulfoxide, N-methylpyrrolidone, or water.

Powders, compositions for broadcasting and dusts can be prepared by mixing or cogrinding of the active substances with a solid carrier.

Granules, for example coated granules, impregnated granules and homogenous granules, can be prepared by binding the active compounds to solid carriers. Solid carriers are, for example, mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as ammonium sulfate, ammonium phosphate, ammonium nitrate, ureas and products of plant origin, such as cereal meal, tree bark meal, wood meal and nutshell meal, cellulose powders and other solid carriers.

The formulations generally comprise from 0.01 to 95% by weight, preferably from 0.1 to 90% by weight, of the active compound. The active compounds are employed in a purity of from 90% to 100%, preferably from 95% to 100% (according to the NMR spectrum).

The following are examples of formulations:

1. Products for Dilution with Water

A) Water-soluble concentrates (SL)
   10 parts by weight of a compound according to the invention are dissolved in water or in a water-soluble solvent. As an alternative, wetters or other auxiliaries are added. The active compound dissolves upon dilution with water.
B) Dispersible concentrates (DC)
   20 parts by weight of a compound according to the invention are dissolved in cyclohexanone with addition of a dispersant, for example polyvinylpyrrolidone. Dilution with water gives a dispersion.
C) Emulsifiable concentrates (EC)
   15 parts by weight of a compound according to the invention are dissolved in xylene with addition of calcium dodecylbenzenesulfonate and castor oil ethoxylate (in each case 5%). Dilution with water gives an emulsion.
D) Emulsions (EW, EO)
   40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.
E) Suspensions (SC, OD)
   In an agitated ball mill, 20 parts by weight of a compound according to the invention are comminuted with addition of dispersants, wetters and water or an organic solvent to give a fine active compound suspension. Dilution with water gives a stable suspension of the active compound.
F) Water-dispersible granules and water-soluble granules (WG, SG)
   50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.
G) Water-dispersible powders and water-soluble powders (WP, SP)
   75 parts by weight of a compound according to the invention are ground in a rotor-stator mill with addition of dispersants, wetters and silica gel. Dilution with water gives a stable dispersion or solution of the active compound.
      2. Products to be Applied Undiluted
H) Dustable powders (DP)
   5 parts by weight of a compound according to the invention are ground finely and mixed intimately with 95% of finely divided kaolin. This gives a dustable product.
I) Granules (GR, FG, GG, MG)
   0.5 part by weight of a compound according to the invention is ground finely and associated with 95.5% carriers. Current methods are extrusion, spray-drying or the fluidized bed. This gives granules to be applied undiluted.
J) ULV solutions (UL)
   10 parts by weight of a compound according to the invention are dissolved in an organic solvent, for example xylene. This gives a product to be applied undiluted.

The active compounds can be applied as such, in the form of their formulations or in the application forms prepared therefrom, for example in the form of directly sprayable solutions, powders, suspensions or dispersions, emulsions, oil dispersions, pastes, dusts, compositions for broadcasting, or granules, by spraying, atomizing, dusting, broadcasting or watering. The application forms depend entirely on the intended uses; in any case, they should ensure very fine dispersion of the active compounds according to the invention.

Aqueous use forms can be prepared from emulsion concentrates, pastes or wettable powders (spray powders, oil dispersions) by addition of water. To prepare emulsions, pastes or oil dispersions, the substances can be homogenized in water as such or dissolved in an oil or solvent, by means of wetting agents, tackifiers, dispersants or emulsifiers. However, concentrates comprising active compound, wetting agent, tackifier, dispersant or emulsifier and possibly solvent or oil which are suitable for dilution with water can also be prepared.

The active compound concentrations in the ready-to-use preparations can be varied over a relatively wide range. In general, they are from 0.0001 to 10%, preferably from 0.01 to 1%.

It is also possible to use the active compounds with a high degree of success in the ultra-low-volume method (ULV), it being possible to apply formulations comprising more than 95% by weight of active compound or even the active compound without additives.

Oils of various types, wetting agents, adjuvants, herbicides, fungicides, other pesticides and bactericides can be added to the active compounds, if desired even immediately prior to application (tank mix). These agents can be added to the compositions according to the invention in a weight ratio of 1:10 to 10:1.

The compositions according to the invention in the use form as fungicides may also be present in combination with other active compounds, for example with herbicides, insecticides, growth regulators, fungicides or else with fertilizers. In many cases, mixing of the compounds I, or of the compositions comprising them, in the use form as fungicides with other fungicides results in a broader fungicidal spectrum of activity.

The following list of fungicides, with which the compounds according to the invention can be used in combination, is intended to illustrate the possible combinations, but not to impose any limitation:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

### Example 1

### Preparation of 2-methylthio-5-chloro-6-(2,4,6-trifluorophenyl)-7-(2-methylbutyl)-1,2,4-triazolo[1,5a]pyrimidine (I-1)

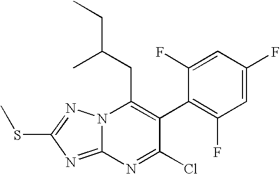

### 1.1. 2-Methylthio-5,7-dihydroxy-6-(2,4,6-trifluorophenyl)-1,2,4-triazolo[1,5a]pyrimidine

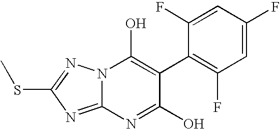

A mixture of 45 g (0.171 mol) of dimethyl (2,4,6-trifluorophenyl)malonate, 30 g (0.162 mol) of tributylamine and 20 g (0.108 mol) of 3-amino-5-methylthio-1,2,4-triazole was stirred at 160° C. for about 6 hours, and the methanol formed was distilled off.

The reaction mixture was then cooled to 80° C., and 100 ml of 20% strength aqueous sodium hydroxide solution were added. The aqueous phase was washed with methyl t-butyl ether, and the organic phase was discarded. The aqueous phase was then acidified with dilute hydrochloric acid and diluted with water, resulting in the precipitation of a lightly colored solid. The solid was stirred overnight, filtered off with suction, washed with water and methyl t-butyl ether and dried at 50° C. under reduced pressure.

This gave 45 g (92%) of compound 1.1. as a beige solid which was used for the next reaction without further purification.

### 1.2. 2-Methylthio-5,7-dichloro-6-(2,4,6-trifluorophenyl)-1,2,4-triazolo[1,5a]pyrimidine

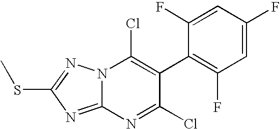

A mixture of 45 g (0.137 mol) of 2-methylthio-5,7-dihydroxy-6-(2,4,6-trifluorophenyl)-1,2,4-triazolo[1,5a]pyrimidine (example 1.1) in 200 ml of phosphorus oxychloride was heated at reflux for about 8 hours.

Excess phosphorus oxychloride was then distilled off, and the residue was taken up in methylene chloride. The organic phase was poured into water and stirred vigorously. The organic phase was separated off, extracted with NaHCO₃ solution and concentrated. The residue was then purified by column chromatography using cyclohexane/ethyl acetate mixtures. This gave 42 g (84%) of the title compound 1.2. as a lightly colored solid.

¹H-NMR (CDCl₃, δ in ppm): 6.9 (t, 2H); 2.75 (s, 3H)

### 1.3. 2-Methylthio-5-chloro-6-(2,4,6-trifluorophenyl)-7-(2-methylbutyl)-1,2,4-triazolo[1,5a]pyrimidine

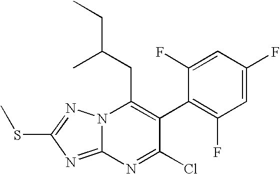

At about 70° C. bath temperature, a solution of 31 g (0.2 mol) of 2-methylbutyl bromide in 50 ml of tetrahydrofuran was added dropwise to a suspension of 4.8 g (0.2 mol) of magnesium and a catalytic amount of iodine in 50 ml of tetrahydrofuran. The mixture was then stirred at this temperature for about 30 min and subsequently cooled to room temperature.

At 10-20° C., this Grignard solution was then added to a mixture of 46 g (0.2 mol) of zinc bromide in 200 ml of tetrahydrofuran. After about 30 min, the mixture was cooled to −10° C. and a mixture of 17.4 g (0.4 mol) of lithium chloride and 18 g (0.2 mol) of copper cyanide in 100 ml of tetrahydrofuran was added. The reaction mixture was then cooled to −40° C., and 36 g (0.1 mol) of 2-methylthio-5,7-dichloro-6-(2,4,6-trifluorophenyl)-1,2,4-triazolo [1,5a]pyrimidine (example 1.2.) in 50 ml of tetrahydrofuran was added. The reaction mixture was stirred at room temperature overnight and then diluted with methyl t-butyl ether and hydrolyzed using ammonium chloride solution.

The organic phase was then separated off, and the aqueous phase was extracted three times with methylene chloride. The combined organic phases were concentrated and the residue was, together with a 12.3 mmol batch which was carried out separately, purified by column chromatography. This gave 39.5 g (88%) of the title compound 1.3. as a lightly colored solid (m.p.=92-94° C.).

¹H-NMR (CDCl₃, δ in ppm): 6.9 (t, 2H); 3.1 (dd, 1H); 2.7 (dd, 1H); 2.7 (s, 3H); 2.05 (m, 1H); 1.25 (m, 1H); 1.15 (m, 1H); 0.8 (t, 3H); 0.75 (d, 3H)

### Example 2

### 2-Methylthio-5-methyl-6-(2,4,6-trifluorophenyl)-7-(2-methylbutyl)-1,2,4-triazolo[1,5a]pyrimidine (I-2)

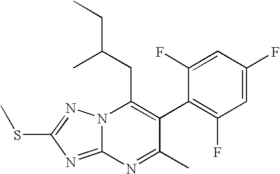

### 2.1. 2-Methylthio-5-(dimethylmalon-2-yl)-6-(2,4,6-trifluorophenyl)-7-(2-methylbutyl)-1,2,4-triazolo[1,5a]pyrimidine

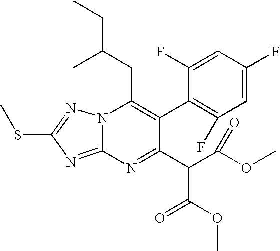

A mixture of 39 g (95 mmol) of 2-methylthio-5-chloro-6-(2,4,6-trifluorophenyl)-7-(2-methylbutyl)-1,2,4-triazolo-[1,5a]pyrimidine (example 1.3) and 40 g (260 mmol) of sodium dimethylmalonate was stirred at a bath temperature of 75° C. for about 3 hours.

The reaction mixture was then cooled to room temperature and methyl t-butyl ether was added, resulting in the precipitation of a yellow solid. This yellow solid was stirred with a mixture of dilute hydrochloric acid and methyl t-butyl ether, resulting in decolorization. The organic phase was then separated off, dried over magnesium sulfate and concentrated. The residue crystallized and was triturated with diisopropyl ether/hexane. This gave 34 g (72%) of the title compound 2.1. as a lightly colored solid (m.p.=126-128° C.).

¹H-NMR (CDCl₃, δ in ppm): 6.9 (t, 2H); 4.7 (s, 1H); 3.75 (s, 3H); 3.7 (s, 3H); 3.0 (dd, 1H); 2.7 (s, 3H); 2.7 (dd, 1H); 2.05 (m, 1H); 1.25 (m, 1H); 1.1 (m, 1H); 0.8 (t, 3H); 0.7 (d, 3H)

### 2.2. 2-Methylthio-5-methyl-6-(2,4,6-trifluorophenyl)-7-(2-methylbutyl)-1,2,4-triazolo[1,5a]pyrimidine

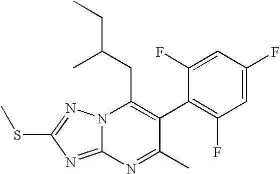

34 g (68 mmol) of 2-methylthio-5-(dimethylmalon-2-yl)-6-(2,4,6-trifluorophenyl)-7-(2-methylbutyl)-1,2,4-triazolo[1,5a]pyrimidine (example 2.1) in 200 ml of concentrated hydrochloric acid were heated at 90° C. for about 4 hours. The reaction mixture was then diluted with water, and the aqueous phase was extracted with methyl t-butyl ether. The combined organic phases were washed with NaHCO₃ solution and water, dried and concentrated. This gave 27 g (quantitative yield) of the title compound 2.2. as a viscous material which slowly crystallized (m.p.=67-69° C.).

¹H-NMR (CDCl₃, δ in ppm): 6.9 (t, 2H); 3.0 (dd, 1H); 2.75 (s, 3H); 2.7 (dd, 1H); 2.4 (s, 3H); 2.05 (m, 1H); 1.3 (m, 1H); 1.15 (m, 1H); 0.8 (t, 3H); 0.75 (d, 3H)

### Example 3

### 2-Methylsulfoxyl-5-methyl-6-(2,4,6-trifluorophenyl)-7-(2-methylbutyl)-1,2,4-triazolo[1,5a]pyrimidine a (1-4) and 2-methylsulfonyl-5-methyl-6-(2,4,6-trifluorophenyl)-7-(2-methylbutyl)-1,2,4-triazolo[1,5a]pyrimidine b (I-3)

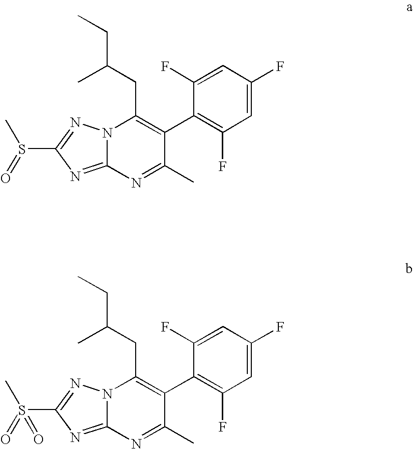

1.6 g (7.2 mmol) of 77% strength m-chloroperbenzoic acid were added to 1.99 g (5 mmol) of 2-methylthio-5-methyl-6-(2,4,6-trifluorophenyl)-7-(2-methylbutyl)-1,2,4-triazolo[1,5a]pyrimidine (example 2.2) in 20 ml of methylene chloride, and the mixture was stirred at room temperature for about 1 hour. Another 100 mg of 77% strength m-chloroperbenzoic acid were then added, and stirring was continued for about 1 hour.

The reaction mixture was then washed with dilute aqueous sodium hydroxide solution and water and concentrated. The residue was purified by column chromatography using cyclohexane/ethyl acetate mixtures.

0.5 g (24%) of the sulfone b and 1.1 g (56%) of the sulfoxide a were obtained in this order as a lightly colored solid and a light yellow oil, respectively.

Sulfone b: (I-3)

¹H-NMR (CDCl₃, δ in ppm): 6.9 (t, 2H); 3.45 (s, 3H); 3.05 (dd, 1H); 2.8 (dd, 1H); 2.5 (s, 3H); 2.05 (m, 1H); 1.25 (m, 1H); 1.1 (m, 1H); 0.8 (t, 3H); 0.75 (d, 2H)

Sulfoxide a: (I-4)

¹H-NMR (CDCl₃, δ in ppm): 6.95 (t, 2H); 3.2 (s, 3H); 3.1 (ddd, 1H); 2.85 (ddd, 1H); 2.5 (s, 3H); 2.05 (m, 1H); 1.25 (m, 1H); 1.15 (m, 1H); 0.75 (m, 6H)

### Example 4

### 2-Cyano-5-methyl-6-(2,4,6-trifluorophenyl)-7-(2-methylbutyl)-1,2,4-triazolo[1,5a]pyrimidine (I-8)

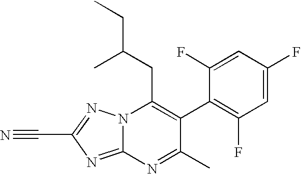

0.8 g (1.94 mmol) of 2-methylsulfonyl-5-methyl-6-(2,4,6-trifluorophenyl)-7-(2-methylbutyl)-1,2,4-triazolo[1,5a]pyrimidine (procedure 3.b) and 2 g (7.5 mmol) of tetrabutylammonium cyanide in 20 ml of acetonitrile were heated at 50° C. for about 7 hours. The reaction mixture was then diluted with water and the aqueous phase was extracted three times with methyl t-butyl ether. The combined organic phases were concentrated and the residue was purified by MPLC on silica gel RP-18 using acetonitrile/water mixtures. This gave 0.5 g (72%) of the title compound 4. as a lightly colored solid (m.p.=113-115° C.).

¹H-NMR (CDCl₃, δ in ppm): 6.95 (t, 2H); 3.05 (dd, 1H); 2.8 (dd, 1H); 2.5 (s, 3H); 2.0 (m, 1H); 1.3 (m, 1H); 1.15 (m, 1H); 0.8 (t, 3H); 0.75 (d, 3H)

### Example 5

### 2-Methoxy-5-methyl-6-(2,4,6-trifluorophenyl)-7-(2-methylbutyl)-1,2,4-triazolo[1,5a]pyrimidine (I-11)

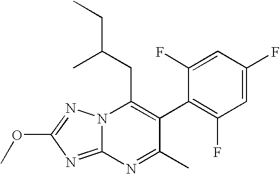

0.83 g (2 mmol) of 2-methylsulfonyl-5-methyl-6-(2,4,6-trifluorophenyl)-7-(2-methylbutyl)-1,2,4-triazolo[1,5a]pyrimidine (example 3.b) and 0.45 g of a 30% strength sodium methoxide solution in 10 ml of acetonitrile were heated at 60° C. for about 7 hours. The reaction mixture was then diluted with water and the aqueous phase was acidified and extracted three times with methyl t-butyl ether. The combined organic phases were concentrated and the residue was purified by MPLC on silica gel RP-18 using acetonitrile/water mixtures. This gave 0.6 g (82%) of the title compound 5. as a yellow oil.

¹H-NMR (CDCl₃, δ in ppm): 6.9 (t, 2H); 4.2 (s, 3H); 2.95 (dd, 1H); 2.65 (dd, 1H); 2.4 (s, 3H); 2.05 (m, 1H); 1.25 (m, 1H); 1.1 (m, 1H); 0.8 (t, 3H); 0.75 (d, 3H)

Table of active compounds

| | |
|---|---|
| | I |
| 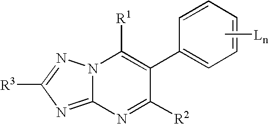 | |

| | | | | | Physical data |
|---|---|---|---|---|---|
| | | | | | (m.p. [° C.], |
| | | | | | IR [cm⁻¹], |
| | | | | | ¹H-NMR |
| No. | R¹ | R² | R³ | Lₙ | [ppm] |
| I-1 | 2-methylbutyl | chloro | S—CH₃ | 2,4,6-F₃ | 92-94 |
| I-2 | 2-methylbutyl | methyl | S—CH₃ | 2,4,6-F₃ | 67-69 |
| I-3 | 2-methylbutyl | methyl | SO₂—CH₃ | 2,4,6-F₃ | 129-130 |
| I-4 | 2-methylbutyl | methyl | SO—CH₃ | 2,4,6-F₃ | 6.95(s, 3H); |
| | | | | | 3.2(s, 3H); |
| | | | | | 2.5(s, 3H) |
| I-5 | 2-methylbutyl | methyl | S-benzyl | 2,4,6-F₃ | 6.85(t, 2H); |
| | | | | | 4.5(s, 2H); |
| | | | | | 2.4(s, 3H) |
| I-6 | 2-methylbutyl | methyl | SO₂-benzyl | 2,4,6-F₃ | 6.9(t, 2H); |
| | | | | | 4.8(s, 2H); |
| | | | | | 2.5(s, 3H) |
| I-7 | 2-methylbutyl | methyl | SO-benzyl | 2,4,6-F₃ | 6.9(t, 2H); |
| | | | | | 2.5(s, 3H); |
| | | | | | 0.65(d, 3H) |
| I-8 | 2-methylbutyl | methyl | cyano | 2,4,6-F₃ | 113-115 |
| I-9 | 2-methylbutyl | methyl | NH-n-C₃H₇ | 2,4,6-F₃ | 126-130 |
| I-10 | 2-methylbutyl | methyl | S—C₂H₅ | 2,4,6-F₃ | 6.85(t, 2H); |
| | | | | | 3.3(q, 2H); |
| | | | | | 2.4(s, 3H) |
| I-11 | 2-methylbutyl | methyl | methoxy | 2,4,6-F₃ | 6.9(t, 2H); |
| | | | | | 4.2(s, 3H); |
| | | | | | 2.4(s, 3H) |
| I-12 | 2-methylbutyl | methyl | ethoxy | 2,4,6-F₃ | 6.9(t, 2H); |
| | | | | | 4.55(q, 2H); |
| | | | | | 2.4(s, 3H) |
| I-13 | 2-methylbutyl | methyl | SO—C₂H₅ | 2,4,6-F₃ | 6.9(s, 2H); |
| | | | | | 3.35-3.5 |
| | | | | | (m, 2H); |
| | | | | | 2.5(s, 3H) |
| I-14 | 2-methylbutyl | methyl | SO₂—C₂H₅ | 2,4,6-F₃ | 6.95(t, 2H); |
| | | | | | 3.6(q, 2H); |
| | | | | | 2.5(s, 3H) |

### USE EXAMPLES

The active compounds were formulated separately as a stock solution comprising 0.25% by weight of active compound in acetone or DMSO. 1% by weight of the emulsifier Uniperol® EL (wetting agent having emulsifying and dispersing action based on ethoxylated alkylphenols) was added to this solution. The stock solutions of the active compounds were diluted with water to the stated concentration.

### Example 1

### Activity Against Gray Mold on Bell Pepper Leaves Caused by Botrytis cinerea, Protective Application

Bell pepper seedlings of the cultivar “Neusiedler Ideal Elite”, were, after 4-5 leaves were well developed, sprayed to runoff point with an aqueous suspension having the concentration of active compound stated below. The next day, the treated plants were inoculated with a spore suspension of Botrytis cinerea which contained 1.7×10⁶ spores/ml in a 2% strength aqueous biomalt solution. The test plants were then placed in a climatized chamber at 22-24° C. and high atmospheric humidity. After 5 days, the extent of the fungal infection on the leaves could be determined visually in %.

In this test, the plants which had been treated with 250 ppm of compound I-2, I-11 or I-12 showed an infection of ≦40%, whereas the untreated (control) plants were 90% infected by the harmful fungus.

### Example 2

### Activity Against Early Blight of Tomato Caused by Alternaria solani

Leaves of potted plants of the cultivar “Golden Princess” were sprayed to runoff point with an aqueous suspension having the concentration of active compound stated below. The next day, the leaves were infected with an aqueous spore suspension of Alternaria solani in 2% biomalt solution having a density of 0.17×10⁶ spore/ml. The plants were then placed in a water-vapor saturated chamber at temperatures between 20 and 22° C. After 5 days, the blight on the untreated, but infected control plants had developed to such an extent that the infection could be determined visually in %.

In this test, the plants which had been treated with 250 ppm of the compound I-11 were <10% infected, whereas the untreated (control) plants were 80% infected.

We claim:

1. A triazolopyrimidine of the formula I 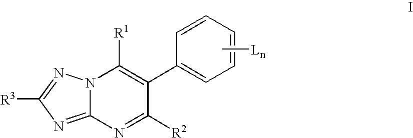
in which the index and the substituents are as defined below:
n is 0 or an integer from 1 to 5;
L is halogen, cyano, hydroxy, cyanato (OCN), C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, a five- to ten-membered saturated, partially unsaturated or aromatic heterocycle which contains 1 to 4 heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
m is 0, 1 or 2;
A,A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl, phenyl, naphthyl, or a five- to ten-membered saturated, partially unsaturated or aromatic heterocycle which is attached via carbon and which contains one to four heteroatoms from the group consisting of O, N and S,
where L and/or R¹ may be partially or fully halogenated or may be substituted by one to four identical or different groups R^{a}:
R^{a} is halogen, cyano, C₁-C₆-alkyl, C₃-C₆-cycloalkyl, C₅-C₆-cycloalkenyl, C₁-C₆-alkoxy, C₁-C₆-alkylthio, C₂-C₆-alkenyl, C₂-C₆-alkenyloxy, C₃-C₆-alkynyloxy, C₂-C₁₀-alkynyl, phenyl, naphthyl, a five- to ten-membered saturated, partially unsaturated or aromatic heterocycle which contains one to four heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
where these aliphatic, alicyclic or aromatic groups for their part may be partially or fully halogenated or may carry one to three groups R^{b}:
R^{b} is halogen, cyano, nitro, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothio-carbonyl, where the alkyl groups in these radicals contain 1 to 6 carbon atoms and the alkenyl or alkynyl groups mentioned contain 2 to 8 carbon atoms in these radicals;
and/or one to three of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups;
R² is C₁-C₄-alkyl, C₂-C₄-alkenyl or C₂-C₄-alkynyl, which may be substituted by halogen, cyano, nitro, C₁-C₂-alkoxy or C₁-C₄-alkoxycarbonyl; and also cyano, chlorine, methoxy; and
R³ is halogen, cyano, C₁-C₈-alkyl, C₁-C₄-haloalkyl, hydroxy, C₁-C₈-alkoxy, C₃-C₈-alkenyl, C₃-C₈-alkenyloxy, C₁-C₈-haloalkoxy, C₃-C₈-haloalkenyloxy, C₃-C₈-cycloalkyl, N(A′)A, N(A′)-C(═O)-A or S(═O)ₘ-A.

2. A triazolopyrimidine as claimed in claim 1
wherein the index and the substituents are as defined below:
L is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
m is 0, 1 or 2;
A,A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl or a five- to ten-membered saturated, partially unsaturated or aromatic heterocycle which is attached via carbon and which contains one to four heteroatoms from the group consisting of O, N and S,
where L and/or R¹ may be partially or fully halogenated or may be substituted by one to four identical or different groups R^{a}:
R^{a} is halogen, cyano, C₁-C₆-alkyl, C₃-C₆-cycloalkyl, C₅-C₆-cycloalkenyl, C₁-C₆-alkoxy, C₁-C₆-alkylthio, C₂-C₆-alkenyl, C₂-C₆-alkenyloxy, C₃-C₆-alkynyloxy, C₂-C₁₀-alkynyl, phenyl, naphthyl, a five- to ten-membered saturated, partially unsaturated or aromatic heterocycle which contains one to four heteroatoms from the group consisting of O, N and S; —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
where these aliphatic, alicyclic or aromatic groups for their part may be partially or fully halogenated or may carry one to three groups R^{b}:
R^{b} is halogen, cyano, nitro, aminocarbonyl, aminothiocarbonyl, alkyl, haloalkyl, alkenyl, alkenyloxy, alkynyloxy, alkoxy, haloalkoxy, alkylamino, dialkylamino, alkylcarbonyl, alkylsulfonyl, alkylsulfoxyl, alkoxycarbonyl, alkylcarbonyloxy, alkylaminocarbonyl, dialkylaminocarbonyl, where the alkyl groups in these radicals contain 1 to 6 carbon atoms and the alkenyl or alkynyl groups mentioned contain 2 to 8 carbon atoms in these radicals;
and/or one to three of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups; and
R² is C₁-C₄-alkyl which may be substituted by halogen, cyano, nitro, C₁-C₂-alkoxy or C₁-C₂-alkoxycarbonyl.

3. A triazolopyrimidine as claimed in claim 2
wherein the index and the substituents are as defined below:
R¹ is C₁-C₁₀-alkyl, C₂-C₁₀-alkenyl, C₂-C₁₀-alkynyl, C₃-C₁₂-cycloalkyl or C₃-C₁₀-cycloalkenyl,
where L and/or R¹ may be partially or fully halogenated or may be substituted by one to four identical or different groups R^{a}:
R^{a} is halogen, cyano, C₁-C₆-alkyl, C₃-C₆-cycloalkyl, C₅-C₆-cycloalkenyl, C₁-C₆-alkoxy, C₁-C₆-alkylthio, C₂-C₆-alkenyl, C₂-C₆-alkenyloxy, C₃-C₆-alkynyloxy, C₂-C₁₀-alkynyl, —C(═O)-A, —C(═O)—O-A, —C(═O)—N(A′)A, C(A′)(═N-OA), N(A′)A, N(A′)-C(═O)-A, N(A″)-C(═O)—N(A′)A, or S(═O)ₘ-A,
where these aliphatic, alicyclic or aromatic groups for their part may be partially or fully halogenated or may carry one to three groups R^{b}:
R^{b} is halogen, cyano, aminocarbonyl, alkyl, haloalkyl, alkenyl, alkenyloxy, alkynyloxy, alkoxy, haloalkoxy, alkylcarbonyl, alkylsulfonyl, alkylsulfoxyl, alkoxycarbonyl, alkylcarbonyloxy, alkylaminocarbonyl, dialkylaminocarbonyl, where the alkyl groups in these radicals contain 1 to 6 carbon atoms and the alkenyl or alkynyl groups mentioned in these radicals contain 2 to 8 carbon atoms;
R² is C₁-C₄-alkyl which may be substituted by halogen, cyano, nitro, C₁-C₂-alkoxy or C₁-C₄-alkoxycarbonyl.

4. A compound of the formula I as claimed in claim 1 in which R³ is S(═O)ₘ-A.

5. A compound of the formula I as claimed in claim 1 in which R³ is SH.

6. A compound of the formula I as claimed in claim 1 in which R² is methyl.

7. A compound of the formula I as claimed in any of claims 1 to 4 in which the phenyl group substituted by Lₙ is the group 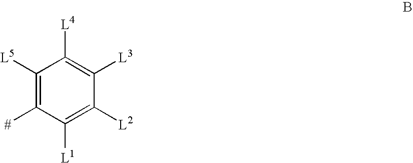
in which # denotes the point of attachment with the triazolopyrimidine skeleton and
L¹ is fluorine, chlorine, CH₃ or CF₃;
L²,L⁴ independently of one another are hydrogen, CH₃ or fluorine;
L³ is hydrogen, fluorine, chlorine, cyano, CH₃, SCH₃, OCH₃, SO₂CH₃, NH—C(═O)CH₃, N(CH₃)—C(═O)CH₃ or COOCH₃ and
L⁵ is hydrogen, fluorine, chlorine or CH₃.

8. A process for preparing the compounds of the formula I as claimed in claim 1 by reacting sulfones of the formula I′ 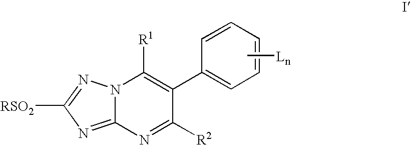
in which R is C₁-C₆-alkyl or unsubstituted or substituted phenyl with compounds of the formula II
(R³)_{y}−M^{y} II
in which R³ has the meaning given for formula I and M is an ammonium, tetraalkylammonium, alkali metal or alkaline earth metal cation and, if R³ is C₁-C₈-alkyl or C₁-C₄-haloalkyl, a metal ion of valency Y selected from the group consisting of: B, Zn, Mg, Si and Sn, under basic conditions.

9. A process for preparing compounds of the formula I as claimed in claim 1 by reacting triazoles of the formula IIIa 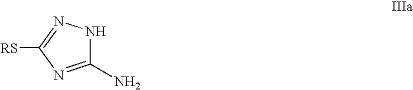
in which R is hydrogen, C₁-C₆-alkyl or unsubstituted or substituted phenyl with dicarbonyl compounds of the formula IV 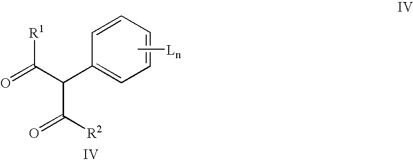
in which n, L, R¹ and R² are as defined in claim 1, to give triazolopyrimidine sulfides of the formula I″ 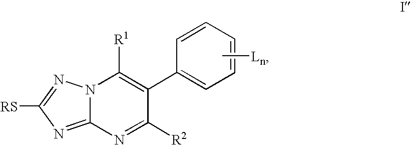
followed, if appropriate, by oxidation of I″ to sulfones of the formula I′ as set forth in claim 8.

10. A composition suitable for controlling harmful fungi, comprising a solid or liquid carrier and a compound of the formula I as claimed in claim 1.

11. A method for controlling phytopathogenic harmful fungi, which comprises treating the fungi or the materials, plants, the soil or the seeds to be protected against fungal attack with an effective amount of the compound of the formula I as claimed in claim 1.
